(19) United States
(12) Patent Application Publication
(10) Pub. No.: US 2023/0364283 A1
(43) Pub. Date: Nov. 16, 2023

(54) DECONTAMINATION DEVICE, DECONTAMINATION ASSEMBLY, DECONTAMINATION SYSTEM, AND RELATED METHODS

(71) Applicant: GERMFLARE APS, Græsted (DK)

(72) Inventor: Flemming T.S. DAHLIN, GRAESTED (DK)

(21) Appl. No.: 18/019,820

(22) PCT Filed: Aug. 5, 2021
(86) PCT No.: PCT/EP2021/071872

§ 371 Date: Feb. 5, 2023

(30) Foreign Application Priority Data
Aug. 6, 2020 (DK) PA202070516
Aug. 6, 2020 (DK) PA202070517
Aug. 6, 2020 (DK) PA202070518
Aug. 6, 2020 (DK) PA202070519
Aug. 6, 2020 (DK) PA202070520
Sep. 4, 2020 (DK) PA202070578
Sep. 4, 2020 (DK) PA202070579

(51) Int. Cl. A61L 2/10; A61L 2/24; A61L 2/26

(52) U.S. Cl. CPC A61L 2/10; A61L 2/24; A61L 2/26; A61L 2202/11; A61L 2202/16

(57) ABSTRACT

A decontamination system, decontamination assembly, decontamination device and related methods are disclosed, the decontamination device comprising a housing comprising a head part and a handle part; a light source configured to emit UV-C light; a light guide for guiding light from the light source towards a UV-transparent area in the head part of the housing; and an interface, wherein the light guide comprises a reflector device having a reflector structure in a proximal surface of the reflector device, the reflector structure comprising a plurality of reflector cavities comprising a first set of reflector cavities and a second set of reflector cavities, each reflector cavity having a reflector surface, the first set of reflector cavities including a first primary reflector cavity having a first primary reflector surface, and a first secondary reflector cavity having a first secondary reflector surface, and the second set of reflector cavities including a second primary reflector cavity having a second primary reflector surface.

### PRIORITY CLAIM

This application is a National Stage Entry based on PCT/EP2021/071872, filed on Aug. 5, 2021, which claims priority to the following Danish Patent Applications:
PA202070517, filed on Aug. 6, 2020;
PA202070516, filed on Aug. 6, 2020;
PA202070518, filed on Aug. 6, 2020;
PA202070519, filed on Aug. 6, 2020;
P 202070520, filed on Aug. 6, 2020;
PA202070578, filed on Sep. 4, 2020; and
PA202070579, filed on Sep. 4, 2020; these applications are incorporated by reference in entirety.

The present disclosure relates to a decontamination device, decontamination assembly, decontamination system and related methods, and in particular to a decontamination device comprising a light guide with a reflector device and being configured to emit UV-C light.

### BACKGROUND

Recently, an increased focus has been developing on how to reduce spreading of vira or other harmful substances or organisms, e.g. by cleaning or decontaminating surfaces or devices in particular due to the outbreak of Covid-19. Hand disinfectants or other liquid cleaning agents may contribute to increased hygiene in turn limiting the spreading of virus and other potentially harmful substances and/or organisms, such as bacteria, fungi, protozoa, algae, germs and/or microbes. However, liquid cleaning agents are in many situations not suitable for disinfection or decontamination, e.g. due to the shape and material of the surface to be disinfected or decontaminated.

### SUMMARY

Accordingly, there is a need for devices, assemblies, systems, and methods, which provide effective and secure tools for improved protection against the spreading of vira, bacteria, or other potentially harmful organisms.

A decontamination device is disclosed, the decontamination device comprising a housing; a light source optionally configured to emit UV-C light and/or UV-A light; a light guide for guiding light from the light source towards a UV-transparent area of the housing, e.g. of a head part of the housing; and an interface.

According to one or more aspects, a decontamination device is disclosed, the decontamination device comprising a housing; a light source optionally configured to emit UV-C light and/or UV-A light; a light guide for guiding light from the light source towards a UV-transparent area of the housing, e.g. of a head part of the housing; and an interface. The light guide optionally comprises a reflector device having a reflector structure in a proximal surface of the reflector device, the reflector structure comprising a plurality of reflector cavities comprising a first set of reflector cavities and/or a second set of reflector cavities. Each reflector cavity optionally has a reflector surface, the first set of reflector cavities including a first primary reflector cavity having a first primary reflector surface, and a first secondary reflector cavity having a first secondary reflector surface, and the second set of reflector cavities optionally including a second primary reflector cavity having a second primary reflector surface.

According to one or more aspects, a decontamination device is disclosed, the decontamination device comprising a housing; a light source optionally configured to emit UV-C light and/or UV-A light; a light guide for guiding light from the light source towards a UV-transparent area of the housing, e.g. of a head part of the housing; and an interface. The light source optionally comprises a circuit board; a first set of UV-C light emitting diodes mounted on the circuit board, the first set of UV-C light emitting diodes optionally comprising at least three UV-C light emitting diodes; and optionally a second set of light emitting diodes mounted on the circuit board, wherein the second set of light emitting diodes is configured to emit visible light and/or UV light comprising UV-A light.

According to one or more aspects, a decontamination device is disclosed, the decontamination device comprising a housing optionally comprising a head part and a handle part; a light source configured to emit UV-C light and/or UV-A light; optionally a light controller connected to the light source for controlling the light source; a light guide for guiding light from the light source towards a UV-transparent area in the head part of the housing; and an interface connected to the light controller. The light controller is configured to control the light source. The decontamination device is optionally configured to operate in one or a plurality of states including a first operating state and optionally one or more of an off state, a lock state, an inactive state, and a second operating state. The light controller optionally comprises an input detector configured to detect one or more inputs of a user via the interface. The input detector may be configured to detect a first input comprising one or a plurality of first input events and the light controller is optionally configured to, in response to a detection of the first input, move the decontamination device to the first operating state.

According to one or more aspects, a decontamination device is disclosed, the decontamination device comprising a housing optionally comprising a head part and a handle part; a light source configured to emit UV-C light and/or UV-A light; a light controller connected to the light source for controlling the light source; a light guide for guiding light from the light source towards a UV-transparent area in the head part of the housing; and an interface connected to the light controller. The light controller is configured to control the light source. The light controller is optionally configured to apply a first light scheme to the light source, wherein to apply the first light scheme optionally comprises to activate a first set of UV-C light emitting diodes of the light source and/or to activate a second set of light emitting diodes of the light source. The second set of light emitting diodes may be configured emit visible light and/or UV light comprising UV-A light.

According to one or more aspects, a decontamination device is disclosed, the decontamination device comprising a housing; a light source optionally configured to emit UV-C light and/or UV-A light; a light guide for guiding light from the light source towards a UV-transparent area of the housing, e.g. of a head part of the housing; and an interface. The decontamination device optionally comprises a heat sink, e.g. as part of a head part, for cooling the light source, the light source comprising: a circuit board made of material comprising a first layer of first material with a first thermal conductivity and a first heat capacity; and optionally a first set of UV-C light emitting diodes mounted on the circuit board. The heat sink may be in thermal contact with the first layer and made of a second material with a second thermal conductivity and a second heat capacity. The second heat capacity is optionally larger than the first heat capacity.

A decontamination assembly is disclosed, the decontamination assembly comprising a decontamination device, e.g. a decontamination device as disclosed herein, and/or a UV shield. Also, a UV shield is disclosed. The decontamination device comprises a housing comprising a head part and a handle part; a light source configured to emit UV-C light and/or UV-A light; a light guide for guiding light from the light source towards a UV-transparent area in the head part of the housing; and an interface, wherein the head part optionally comprises a coupling device for releasably coupling the head part to a port of the UV shield.

Further, a decontamination system is disclosed. The decontamination system comprising a container having an inner volume defined by at least one wall including a first wall. The decontamination system may comprise a carrier device optionally rotatably arranged about a rotation axis in the inner volume; a first port in the first wall, wherein the first port is configured to couple a decontamination device to the container; and optionally a second port in a wall, such as the first wall or a second wall, of the container, wherein the second port is optionally configured to couple a decontamination device to the container.

The decontamination system optionally comprises a motor configured to rotate the carrier device. The carrier device may be translationally arranged along and/or perpendicular to the rotation axis. The decontamination system may comprise one or more decontamination devices as disclosed herein.

The present disclosure provides a decontamination device, decontamination assembly or system that is safe and efficient to use. For example, the present disclosure significantly reduces the time needed for preparing or warming up the device. Further, the disclosed decontamination device reduces the duration of a decontamination procedure, and in particular the required exposure time.

Further, the present disclosure provides devices, assemblies, systems, and associated methods, that allow for a safe decontamination of surfaces or devices, such as door handles, elevator user interfaces, mobile telephones, tablets, headsets, eyewear, steering wheels, and keyboards.

In addition, UV-C light and/or UV-A can be directed and controlled towards a desired item to be decontaminated.

The present disclosure may provide a safe decontamination device, e.g. protected against unauthorized or accidental use. Further, light scheme(s) as disclosed herein allows for easy design and configuration of the decontamination device to different applications and/or effective decontamination of items.

The present disclosure provides a decontamination device with improved heat distribution capabilities in turn providing an effective and safe decontamination device and/or a decontamination device with longer component lifetime.

### BRIEF DESCRIPTION OF THE DRAWINGS

The above and other features and advantages of the present disclosure will become readily apparent to those skilled in the art by the following detailed description of example embodiments thereof with reference to the attached drawings, in which:

FIG. 1A is a first perspective view of an exemplary decontamination device,

FIG. 1B is a second perspective view of an exemplary decontamination device,

FIG. 2 is an exploded view of an exemplary decontamination device,

FIG. 3A is a cross-sectional exploded view of an exemplary decontamination device,

FIG. 3B is a cross-sectional view of an exemplary decontamination device,

FIG. 4 is a schematic block diagram of an exemplary decontamination device,

FIG. 5A is a perspective view of an exemplary light source,

FIG. 5B is a cross sectional view along the axis A of example light sources,

FIG. 5C is a perspective view of an exemplary light source,

FIG. 5D is a perspective view of an exemplary light source,

FIG. 6A is a proximal view of an exemplary light guide showing a proximal surface of the light guide,

FIG. 6B is a perspective cross-sectional perspective view of an exemplary light guide seen from a proximal side,

FIG. 6C is a perspective view of an exemplary light guide seen from the distal side,

FIG. 6D is a cross-sectional view of an exemplary light guide along the axis B shown in FIG. 6A,

FIG. 7A is a perspective view of an exemplary spacer ring,

FIG. 7B is a perspective view of an exemplary spacer plate,

FIG. 8A is a proximal view of an exemplary coupling ring,

FIG. 8B is a perspective view of an exemplary coupling ring,

FIG. 9A is a perspective view of an exemplary decontamination assembly comprising a first type of UV shield,

FIG. 9B is a side-view of an exemplary decontamination assembly comprising a first type of UV shield,

FIG. 10A is a proximal view of an exemplary decontamination assembly,

FIG. 10B is a proximal view of an exemplary UV shield,

FIG. 11 is an exploded perspective view of an exemplary decontamination assembly,

FIG. 12A is a side-view of an exemplary decontamination assembly comprising a second type of UV shield,

FIG. 12B is a perspective view of an exemplary decontamination assembly comprising a second type of UV shield,

FIG. 13A is a side-view of an exemplary decontamination assembly comprising a third type of UV shield,

FIG. 13B is a perspective view of an exemplary decontamination assembly comprising a third type of UV shield,

FIG. 14 is a perspective view of an exemplary decontamination system,

FIG. 15 is a schematic view of an exemplary decontamination system,

FIG. 16 is a timing diagram of an exemplary first light scheme,

FIG. 17 is a timing diagram of an exemplary first light scheme,

FIG. 18 shows a state diagram of the light controller, and

FIG. 19 shows a state diagram of the light controller.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The figures are schematic and simplified for clarity, and they merely show details which aid understanding the disclosure, while other details have been left out. Throughout, the same reference numerals are used for identical or corresponding parts.

In the following, whenever referring to proximal side or surface of a layer, an element, a device or part of a device, the referral is to the side closest to or the surface facing a surface to be decontaminated, when the decontamination device is in use. Likewise, whenever referring to the distal side or surface of a layer, an element, a device or part of a device, the referral is to the side furthest away from or the surface facing away from the surface to be decontaminated, when the decontamination device is in use. In other words, the proximal side or surface is the side or surface closest to the surface to be decontaminated, when the decontamination device is in use and the distal side is the opposite side or surface—the side or surface furthest away from the surface to be decontaminated in use.

A radial direction is defined as perpendicular to a longitudinal axis or direction of the decontamination device. In some sentences, the words “inner” and “outer” may be used. These qualifiers should generally be perceived with respect to the radial direction, such that a reference to an “outer” element means that the element is farther away from a center portion of the decontamination device than an element referenced as “inner”. In addition, “innermost” should be interpreted as the portion of a component forming a center of the component and/or being adjacent to the center of the component. In analogy, “outermost” should be interpreted as a portion of a component forming an outer edge or outer contour of a component and/or being adjacent to that outer edge or outer contour.

In the present context, when referring to emitting light with a power of e.g. 10 mW, it is understood that the emitted light has optical power of 10 mW.

A decontamination device is provided. Decontamination device may be understood as a device that is configured to decontaminate a surface from one or more harmful substances and/or organisms, such as virus, bacteria, fungi, protozoa, algae, germs and/or microbes. Further, decontamination device may be understood as a device configured to make a surface, such as an object or an area, safe for unprotected people, by removing, neutralizing, and/or destroying one or more harmful substances and/or organisms. The decontamination device may additionally or alternatively be denoted as a germicidal device, a disinfection device, a sterilization device, a cleaning device, a purifying device, antibacterial device, hygienizing device, and/or sanitizing device.

A decontamination device comprising a housing; a light source configured to emit UV-C light and/or UV-A light; a light controller connected to the light source for controlling the light source; a light guide for guiding light from the light source towards a UV-transparent area in the head part of the housing; and an interface connected to the light controller.

In one or more exemplary decontamination devices, the light source comprises a first set of UV-C light emitting diodes of the light source and/or a second set of light emitting diodes optionally configured to emit visible light and/or UV light comprising UV-A light.

The light controller is configured to apply one or more light schemes, e.g. a first light scheme and/or a second light scheme, to the light source, e.g. in accordance with a user input via the interface.

The light controller is optionally configured to, e.g. in the first operating state, apply a first light scheme to the light source, wherein to apply the first light scheme comprises to activate the first set of UV-C light emitting diodes (LEDs) of the light source and/or to activate the second set of light emitting diodes (LEDs) of the light source. In other, words, the first set of UV-C LEDs is optionally activated and therefore emits UV-C light when the first light scheme is applied in the light source. Further the second set of light emitting diodes of the light source is optionally activated and therefore emits visible light and/or UV light comprising UV-A light when the first light scheme is applied in the light source.

The decontamination device is optionally configured to operate in one or a plurality of states including a first operating state and optionally one or more of an off state, a lock state, an inactive state, and a second operating state.

The first operating state may be characterized by the light source being activated and/or turned on, e.g. according to the first light scheme. In other words, the first operating state may be characterized by the light controller applying the first light scheme in the light source.

The second operating state may be characterized by the light source being activated and/or turned on, e.g. according to the second light scheme. In other words, the second operating state may be characterized by the light controller applying the second light scheme in the light source.

The off state may be characterized in that the power consumption in the decontamination device is zero or less than an off threshold.

The lock state may be characterized in that a light source in the second interface is activated and/or that the light source or at least UV-C LEDs of the light and/or UV_A LEDs of the light source are off or dimmed.

The inactive state may be characterized in that a light source in the second interface is activated and/or that visible light is emitted from the light source comprising UV LEDs. For example, UV_A LEDs also emitting visible light may be activated at reduced power compared to the first light scheme and/or the second light scheme in the inactive state. In one or more exemplary decontamination devices, the light controller/input detector may not detect first input and/or second input in a pause period after moving to the inactive state. Thereby it may be ensured that the LEDs are not overheated or that the LEDs have sufficient time to cool off before being activated again, in turn leading to longer life time of the decontamination device.

The decontamination device, such as the light controller and/or the interface, may comprise an input detector configured to detect one or more inputs including a first input and/or a second input, e.g. in inactive state, lock state or off state. The input detector is configured to detect different inputs including the first input and/or the second input based on one or more input signals from interface. An input signal to the input detector may be indicative of one or more of a button press, a button push-and-hold, a rotation, such as a left rotation and/or a right rotation, and a slide, such as in a first direction and/or a second direction.

An input comprises one or a plurality of input events, e.g. the first input optionally comprises one or a plurality of first input events. The second input optionally comprises one or a plurality of second input events. The first input is optionally different from the second input.

In one or more exemplary decontamination devices, the first input comprises a first primary input event and optionally a first secondary input event. The first primary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release. The first secondary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release. A first input requiring a plurality of input events and/or multi-input events increases safety by reducing the risk of unauthorized use, accidental activation and/or a child activating the decontamination device.

In one or more exemplary decontamination devices, the first input comprises a first tertiary input event. The first tertiary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release.

In one or more exemplary decontamination devices, the first input comprises a first quaternary input event. The first quaternary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release.

In one or more exemplary decontamination devices, the first input comprises at least three input events, such as first primary input event, first secondary input event, and first tertiary input event. In one or more exemplary decontamination devices, the number of first input events of the first input is in the range from three to five, providing a sufficiently complex activation and on the other hand not being too hard to remember.

The first primary input event and/or the first secondary input event may be a first rotate-and-release input. The first rotate-and-release input may comprise rotating a rotation ring element of the interface in a first direction, such as a left direction, optionally about the central axis.

The first primary input event and/or the first secondary input event may be a second rotate-and-release input. The second rotate-and-release input may comprise rotating a rotation ring element of the interface in a second direction, such as a right direction, optionally about the central axis.

The first tertiary input event and/or the first quaternary input event may be a first rotate-and-release input. The first rotate-and-release input may comprise rotating a rotation ring element of the interface in a first direction, such as a left direction, optionally about the central axis.

The first tertiary input event and/or the first quaternary input event may be a second rotate-and-release input. The second rotate-and-release input may comprise rotating a rotation ring element of the interface in a second direction, such as a right direction, optionally about the central axis.

The first primary input event and the first secondary input event may be different. The first primary input event and the first tertiary input event may be different.

Exemplary first inputs A_1-I_1 are outlined in the table below, where FPIE is the first primary input event, FSIE is the first secondary input event, FTIE is the first tertiary input event, and FQIE is the first quaternary input event. Further, I_R_R_1 is a first rotate-and-release input with rotation in first direction, I_R_R_2 is a second rotate-and-release input with rotation in second direction, I_R_H_R_1 is a first rotate-hold-and-release input with rotation in first direction, and I_R_H_R_2 is a second rotate-hold-and-release input with rotation in second direction, I_P_R is a press-and-release input, e.g. on a button of the interface.

TABLE 1
Exemplary first inputs A_1-I_1

| | FPIE | FSIE | FTIE | FQIE |
|---|---|---|---|---|
| A_1 | I_R_R_1 | I_R_R_1 | N/A | N/A |
| B_1 | I_R_R_1 | I_R_R_2 | I_R_R_1 | I_R_R_2 |
| C_1 | I_R_R_1 | I_R_R_1 | I_R_R_2 | I_R_R_2 |
| D_1 | I_R_R_1 | I_R_R_1 | I_R_R_1 | I_R_R_1 |
| | | | | or N/A |
| E_1 | I_R_H_R_1 | I_R_H_R_2 | N/A | N/A |
| F_1 | I_P_R | I_P_R | I_P_R | N/A |
| G_1 | I_P_R | I_R_R_1 | N/A | N/A |
| H_1 | I_R_H_R_1 | I_R_R_1 | I_R_R_1 or | N/A |
| | | | N/A | |
| I_1 | I_R_R_1 | I_R_R_1 | I_R_H_R_1 | N/A |

In one or more exemplary decontamination devices, the first input comprises one or more rotation input events and one or more press input events. The first input may have a duration less than 5 seconds or even less than 3 seconds. In other words, the first input may be detected within a first input period less than 5 seconds, e.g. less than 3 seconds.

An input event, such as a rotate-and-release input event and/or a press-and-release input event, may have a duration less than 100 ms. An input event, such as a rotate-hold-and-release input event and/or a press-hold-and-release input event, may have a duration of more than 100 ms. Other durations may be applied depending on the desired input detection.

The light controller is configured to, in response to a detection of input, move the decontamination device between the states depending on the input and/or the present state. For example, the light controller is optionally configured to, in response to a detection of the first input, move the decontamination device to the first operating state. In one or more exemplary decontamination devices, the light controller is configured to move the decontamination device to the first operating state from one or more of the off state, the lock state and/or the inactive state in response to a detection of the first input, i.e. in response to the input detector detecting the first input.

In one or more exemplary decontamination devices, the input detector is configured to detect an unlock input. The light controller is optionally configured to, in response to detection of the unlock input, to move the decontamination device to the inactive state, e.g. from the off state and/or the lock state. In other words, the light controller may be configured to, when the decontamination device is in the off state and/or the lock state and when an unlock input is detected, move the decontamination device to the inactive state.

The unlock input may consist of a single unlock input event or two unlock input events. Thereby, the decontamination device may provide a simple unlock functionality. The unlock input may comprise or consist of a button press input and/or a rotate-and-release input, such as a first rotate-and-release input or a second rotate-and-release input. The unlock input may comprise or consist of a first rotate-and-release input (first unlock input event) optionally followed by a second rotate-and-release input (second unlock input event) or vice versa. The unlock input may have a duration less than 5 seconds or even less than 3 seconds. In other words, the unlock input may be detected within an unlock input period less than 5 seconds, e.g. less than 3 seconds.

In one or more exemplary decontamination devices, the input detector is configured to detect a lock input, e.g. in one or more of the inactive state, the first operating state, and the second operating state. The light controller is optionally configured to, in response to detection of the lock input, move the decontamination device to the lock state, e.g. from one or more of the inactive state, the first operating state, and the second operating state.

The lock input may consist of a single lock input event or two lock input events. Thereby, the decontamination device may provide a simple lock functionality. The lock input event may comprise or consist of a button press input and/or a rotate-and-release input, such as a first rotate-and-release input or a second rotate-and-release input. The lock input may comprise or consist of a first rotate-and-release input (first lock input event) optionally followed by a second rotate-and-release input (second lock input event) or vice versa. The lock input may have a duration less than 5 seconds or even less than 3 seconds. In other words, the lock input may be detected within a lock input period less than 5 seconds, e.g. less than 3 seconds.

In one or more exemplary decontamination devices, the input detector is configured to detect an off input. The light controller is optionally configured to, in response to detection of the off input, move the decontamination device to the off state, e.g. from one or more of the inactive state, the first operating state, and the second operating state.

The off input may consist of a single off input event or two off input events. Thereby, the decontamination device may provide a simple off functionality. The off input event may comprise or consist of a button press input and/or a rotate-and-release input, such as a first rotate-and-release input or a second rotate-and-release input. The off input may comprise or consist of a first rotate-and-release input (first off input event) optionally followed by a second rotate-and-release input (second off input event) or vice versa. The off input may have a duration less than 5 seconds or even less than 3 seconds. In other words, the off input may be detected within an off input period less than 5 seconds, e.g. less than 3 seconds.

In one or more exemplary decontamination devices, the input detector is configured to detect an on input, e.g. in the off state. The light controller is optionally configured to, in response to detection of the on input, move the decontamination device to the lock state, the inactive state, the first operating state, or the second operating state.

The on input may consist of a single on input event or two on input events. Thereby, the decontamination device may provide a simple on functionality, e.g. without applying a light scheme. The on input may comprise three, four, or more on input events. The on input event may comprise or consist of a button press input and/or a rotate-and-release input, such as a first rotate-and-release input or a second rotate-and-release input. The on input may comprise or consist of a first rotate-and-release input (first on input event) optionally followed by a second rotate-and-release input (second on input event) or vice versa. The on input may have a duration less than 5 seconds or even less than 3 seconds. In other words, the on input may be detected within an on input period less than 5 seconds, e.g. less than 3 seconds.

In one or more exemplary decontamination devices, the light controller is configured to, in the first operating state, apply the first light scheme. The light controller may be configured to, after applying the first light scheme, move to the off state, the inactive state or the lock state. In other words, the decontamination device may apply the first light scheme in the first operating state and move to the off state, the inactive state or the lock state after application of the first light scheme.

In one or more exemplary decontamination devices, the light controller is configured to, in the second operating state, apply the second light scheme. The light controller may be configured to, after applying the second light scheme, move to the off state, the inactive state or the lock state. In other words, the decontamination device may apply the second light scheme in the second operating state and move to the off state, the inactive state or the lock state after application of the second light scheme.

In one or more exemplary decontamination devices, the light controller is configured to, in the inactive state, turn off the light source and/or activate, e.g. including strobing optionally with fixed, increasing or decreasing duty cycle, a light source of the second interface.

In one or more exemplary decontamination devices, the light controller is configured to, in the lock state, turn off the light source and/or activate, e.g. including strobing optionally with fixed, increasing or decreasing duty cycle, a light source of the second interface. To activate, e.g. including strobing optionally with fixed, increasing or decreasing duty cycle, a light source of the second interface in the lock state may comprise to activate the light source of the second interface different compared to the inactive state, e.g. by constant light in the lock state and strobing in the inactive state or vice versa, or by strobing with different duty cycles in the lock state and the inactive state. Thus, the internal operating state of the decontamination device may be communicated to the user in turn securing a safe operation of the decontamination device. In one or more exemplary decontamination devices, the light source of the second interface may be configured to emit a plurality of colors, e.g. where a lock color, such as red, blue, or green, indicates the lock state and/or where an inactive color, such as red, blue or green, optionally different from the lock color indicates the inactive state.

In one or more exemplary decontamination devices, the light controller is configured to, in the inactive state, turn off the light source and/or activate, e.g. including strobing optionally with fixed, increasing or decreasing duty cycle, a light source of the second interface. Thus, the internal operating state of the decontamination device may be communicated to the user in turn securing a safe operation of the decontamination device.

In one or more exemplary decontamination devices, the input detector is configured to detect a second input. The light controller is optionally configured to, in response to a detection of the second input, move the decontamination device to a second operating state and optionally apply, in the second operating state, a second light scheme. The light controller is optionally configured to, after applying the second light scheme, move the decontamination device to the inactive state, the off state or the lock state.

In one or more exemplary decontamination devices, the second input comprises a second primary input event and optionally a second secondary input event. The second primary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release. The second secondary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release. A second input requiring a plurality of input events and/or multi-input events increases safety by reducing the risk of unauthorized use, accidental activation and/or a child activating the decontamination device. In one or more exemplary decontamination devices, the second input has fewer or more input events than the first input. In other words, the number of second input events of the second input may be less or larger than the number of first input events of the first input. Thus, a more complex second input may be required for activation of the second light scheme, e.g. in case the second light scheme is more dangerous or implies a higher risk to the user than the first light scheme.

In one or more exemplary decontamination devices, the second input comprises a second tertiary input event. The second tertiary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release.

In one or more exemplary decontamination devices, the second input comprises a second quaternary input event. The second quaternary input event optionally comprises one or more of a press-and-hold input, a press input, a multi-press input, a rotate-and-hold input, a rotate input, a multi-rotate input, a slide-and-hold input, a slide input, and a multi-slide input, e.g. followed by release.

In one or more exemplary decontamination devices, the second input comprises at least three input events, such as second primary input event, second secondary input event, and second tertiary input event. In one or more exemplary decontamination devices, the number of second input events of the second input is in the range from three to five, providing a sufficiently complex activation and on the other hand not being too hard to remember.

The second primary input event and/or the second secondary input event may be a first rotate-and-release input. The first rotate-and-release input may comprise rotating a rotation ring element of the interface in a first direction, such as a left direction, optionally about the central axis.

The second primary input event and/or the second secondary input event may be a second rotate-and-release input. The second rotate-and-release input may comprise rotating a rotation ring element of the interface in a second direction, such as a right direction, optionally about the central axis.

The second tertiary input event and/or the second quaternary input event may be a first rotate-and-release input. The first rotate-and-release input may comprise rotating a rotation ring element of the interface in a first direction, such as a left direction, optionally about the central axis.

The second tertiary input event and/or the second quaternary input event may be a second rotate-and-release input. The second rotate-and-release input may comprise rotating a rotation ring element of the interface in a second direction, such as a right direction, optionally about the central axis.

The second primary input event and the second secondary input event may be different. The second primary input event and the second tertiary input event may be different.

Exemplary second inputs A_2-I_2 K are outlined in the table below, where SPIE is the second primary input event, SSIE is the second secondary input event, STIE is the second tertiary input event, and SQIE is the second quaternary input event. Further, I_R_R_1 is a first rotate-and-release input with rotation in first direction, I_R_R_2 is a second rotate-and-release input with rotation in second direction, I_R_H_R_1 is a first rotate-hold-and-release input with rotation in first direction, and I_R_H_R_2 is a second rotate-hold-and-release input with rotation in second direction, I_P_R is a press-and-release input, e.g. on a button of the interface.

TABLE 2
Exemplary second inputs A_2-I_2

| | SPIE | SSIE | STIE | SQIE |
|---|---|---|---|---|
| A_2 | I_R_R_2 | I_R_R_2 | N/A | N/A |
| B_2 | I_R_R_2 | I_R_R_1 | I_R_R_2 | I_R_R_1 |
| C_2 | I_R_R_2 | I_R_R_2 | I_R_R_1 | I_R_R_{—} |
| D_2 | I_R_R_2 | I_R_R_2 | I_R_R_2 | I_R_R_2 |
| | | | | or N/A |
| E_2 | I_R_H_R_2 | I_R_H_R_1 | N/A | N/A |
| F_2 | I_P_R | N/A | N/A | N/A |
| G_2 | I_P_R | I_R_R_2 | N/A | N/A |
| H_2 | I_R_H_R_2 | I_R_R_2 | I_R_R_2 or | N/A |
| | | | N/A | |
| I_2 | I_R_R_2 | I_R_R_2 | I_R_H_R_2 | N/A |

In one or more exemplary decontamination devices, the second input comprises one or more rotation input events and one or more press input events. The second input may have a duration less than 5 seconds or even less than 3 seconds. In other words, the second input may be detected within a second input period less than 5 seconds, e.g. less than 3 seconds.

In one or more exemplary decontamination devices, the light controller is configured to reset a timer when moving the decontamination device to a state, e.g. the inactive state, and move the decontamination device to another state, e.g. to the lock state from the inactive state, if the timer reaches a time threshold. The time threshold may be in the range from 5 seconds to 3 minutes, such as in the range from 5 seconds to 1 minute, e.g. 10 seconds, 20 seconds, 30 seconds. Thereby, the safety may be further increased to reduce the risk of accidental activation.

In one or more exemplary decontamination devices, the light controller is configured to reset a timer when moving the decontamination device to the lock state, and optionally move the decontamination device to the off state from the lock state, if the timer reaches a time threshold. The time threshold may be in the range from 5 seconds to 10 minutes, such as in the range from 30 seconds to 5 minutes. Thereby, power consumption may be reduced and/or accidental on input by a user may be cancelled.

In one or more exemplary decontamination devices, the input detector is configured to detect a stop input, e.g. in the first operating state and/or in the second operating state, and wherein the light controller optionally is configured to, in response to detection of the stop input, deactivate the light source and/or move the decontamination device to the inactive state, the lock state or the off state.

The stop input may consist of a single stop input event or two stop input events. Thereby, the decontamination device may provide a simple (emergency) stop functionality, e.g. without applying a light scheme. The stop input may comprise three, four, or more stop input events. The stop input event may comprise or consist of a button press input and/or a rotate-and-release input, such as a first rotate-and-release input or a second rotate-and-release input. The stop input may comprise or consist of a first rotate-and-release input (first stop input event) optionally followed by a second rotate-and-release input (second stop input event) or vice versa. The stop input may have a duration less than 5 seconds or even less than 3 seconds. In other words, the stop input may be detected within an stop input period less than 5 seconds, e.g. less than 3 seconds, or even less than 1 second.

In one or more exemplary decontamination devices, the light controller is configured to apply a second light scheme to the light source, e.g. in the second operating state. The second light scheme is optionally different from the first light scheme.

In one or more exemplary decontamination devices, to apply the second light scheme comprises only to activate the first set of UV-C light emitting diodes (LEDs) of the light source. In other words, the second set of LEDs (and/or other LEDs) may be off or dimmed in the second light scheme. In one or more exemplary decontamination devices, to apply the second light scheme comprises only to activate the second set of LEDs. In other words, the first set of LEDs (and/or other LEDs) may be off or dimmed in the second light scheme. This allows for different applications of the decontamination device to different tasks heavily increasing the user flexibility of the decontamination device.

In one or more exemplary decontamination devices, to activate the first set of UV-C light emitting diodes of the light source, e.g. in the first light scheme and/or in the second light scheme, comprises strobing the first set of UV-C light emitting diodes. In one or more exemplary decontamination devices, strobing the first set of UV-C light emitting diodes comprises operating the first set of UV-C light emitting diodes with a first duty cycle having a first period, e.g. in the range from 1 ms to 100 ms, such as in the range from 5 ms to 30 ms, e.g. about 10 ms, 15 ms, or 20 ms. The first duty cycle may be larger than 20%, e.g. in the range from 25% to 95%, such as in the range from 40% to 80%. Running/operating the first set of LEDs with a duty cycle may reduce the cooling requirements of the light source for a given output and/or facilitate a higher light output at a given power consumption. Further, the lifetime of the first set of LEDs may be increased. Thus, a more robust and power efficient decontamination device is provided. Further, the overall decontamination efficiency of the decontamination device may be increased.

In one or more exemplary decontamination devices, the first light scheme has a duration less than 2 minutes, e.g. less than 1 minute or even in the range from 1 second to 30 seconds. Thereby, a fast decontamination of an item is allowed for heavily increasing the user friendliness of the device. Also, a safe operation of the decontamination device is allowed by reducing the risk of undesired items being exposed to UV-C light. The first light scheme may have a duration in the range from 1 second to 15 seconds.

In one or more exemplary decontamination devices, the second set of light emitting diodes comprises a plurality of UV-A light emitting diodes. To activate the second set of light emitting diodes of the light source, e.g. in the first light scheme and/or in the second light scheme, optionally comprises strobing the second set of light emitting diodes. In one or more exemplary decontamination devices, strobing the second set of light emitting diodes comprises operating the second set of light emitting diodes with a second duty cycle having a second period, e.g. in the range from 1 ms to 100 ms, such as in the range from 5 ms to 30 ms, e.g. about 10 ms, 15 ms, or 20 ms. The second duty cycle may be larger than 20%, e.g. in the range from 25% to 95%, such as in the range from 40% to 80%. Running/operating the second set of LEDs with a duty cycle may reduce the cooling requirements of the light source for a given output and/or facilitate a higher light output at a given power consumption. Further, the lifetime of the second set of LEDs may be increased. Thus, a more robust and power efficient decontamination device is provided. Further, the overall decontamination efficiency of the decontamination device may be increased.

In one or more exemplary decontamination devices, the first pulse of the first duty cycle is shifted in relation to the second pulse of the second duty cycle, e.g. the first pulse (or first active time) of the first duty cycle may be during the second off time (or second deactive time) of the second duty cycle and/or the second pulse (or second active time) of the second duty cycle may be during the first off time (or first deactive time) of the first duty cycle. This may lead to an improved heat distribution in the decontamination device and/or more evenly distributed load on the power source.

In one or more exemplary decontamination devices, the second light scheme has a duration less than 2 minutes, e.g. less than 1 minute or even in the range from 1 second to 30 seconds. Thereby, a fast decontamination of an item is allowed for heavily increasing the user friendliness of the device. Also, a safe operation of the decontamination device is allowed by reducing the risk of undesired items being exposed. The second light scheme may have a duration in the range from 1 second to 15 seconds.

In one or more exemplary decontamination devices, to apply the first light scheme and/or the second light scheme comprises to activate a third light emitting diode of the light source configured to emit visible light.

In one or more exemplary decontamination devices, to activate the first set of UV-C light emitting diodes of the light source, e.g. in the first light scheme and/or in the second light scheme, comprises to activate, e.g. including strobing with a first duty cycle, the first set of UV-C light emitting diodes for a first primary time period. The first duty cycle may be constant or vary during the first light scheme and/or in the second light scheme

In one or more exemplary decontamination devices, to activate the second set of light emitting diodes of the light source, e.g. in the first light scheme and/or in the second light scheme, comprises to activate, e.g. including strobing with a second duty cycle, the second set of light emitting diodes for a second primary time period. The second duty cycle may be constant or vary during the first light scheme and/or in the second light scheme

In one or more exemplary decontamination devices, the second primary time period may start before or at the same time as the start of the first primary time period. Thus, visible light may be emitted before and/or during UV-C light being emitted in turn notifying the user on an upcoming UV-C light emission. Thus, the safety for the user is increased.

In one or more exemplary decontamination devices, the first light scheme and/or the second light scheme, may comprise a first primary pause and/or a first visible time period before the first primary time period. The light controller may be configured to, in a first visible time period of the first light scheme and/or the second light scheme, activate, e.g. including strobing optionally with fixed, increasing or decreasing duty cycle, the second set of LEDs (second duty cycle) and/or the third LED (third duty cycle). The light controller may be configured to, in a first visible time period of the first light scheme and/or the second light scheme, activate, e.g. including strobing optionally with fixed, increasing or decreasing duty cycle, a light source of the second interface. Thereby, a user can be provided with information about the operation of the decontamination device, and in turn enable providing an indication to the user of an internal state of the decontamination device and thus the remaining time frame before UV-C light (which is not visible and may be dangerous to the user) is emitted. Thereby, the operation safety of the decontamination device is increased.

In one or more exemplary decontamination devices, the first primary time period is less than 1 second or in the range from 1 second to 10 seconds. In one or more exemplary decontamination devices, the first primary time period is less than 2 minutes, e.g. less than 1 minute or even in the range from 1 second to 30 seconds. The first primary time period may be in the range from 1 second to 15 seconds.

In one or more exemplary decontamination devices, the second primary time period is less than 1 second or in the range from 1 second to 10 seconds. In one or more exemplary decontamination devices, the second primary time period is less than 2 minutes, e.g. less than 1 minute or even in the range from 1 second to 30 seconds. The second primary time period may be in the range from 1 second to 15 seconds.

The first primary time period may overlap or be separate from the second primary time period. The first primary time period may be before the second primary time period. The first primary time period may be after the second primary time period.

In one or more exemplary decontamination devices, to activate the first set of UV-C light emitting diodes of the light source comprises to activate the first set of UV-C light emitting diodes for a first secondary time period, e.g. after the second primary time period. In one or more exemplary decontamination devices, the first secondary time period is less than 1 second or in the range from 1 second to 10 seconds. In one or more exemplary decontamination devices, the first secondary time period is less than 2 minutes, e.g. less than 1 minute or even in the range from 1 second to 30 seconds. The first secondary time period may be in the range from 1 second to 15 seconds.

In one or more exemplary decontamination devices, to activate the second set of light emitting diodes of the light source comprises to activate the second set of light emitting diodes for a second secondary time period, e.g. after the first secondary time period. In one or more exemplary decontamination devices, the second secondary time period is less than 1 second or in the range from 1 second to 10 seconds. In one or more exemplary decontamination devices, the second secondary time period is less than 2 minutes, e.g. less than 1 minute or even in the range from 1 second to 30 seconds. The second secondary time period may be in the range from 1 second to 15 seconds.

In one or more exemplary decontamination devices, to activate the first set of UV-C light emitting diodes of the light source comprises to emit UV-C light with a power larger than 10 mW, such as in the range from 20 mW to 800 mW. To activate the first set of UV-C light emitting diodes of the light source optionally comprises to emit UV-C light with a power in the range from 1 mW to 20 mW or in the range from 20 mW to 50 mW. In one or more exemplary decontamination devices, to activate the first set of UV-C light emitting diodes of the light source optionally comprises to emit UV-C light with a power in the range from 50 mW to 1,000 mW or even larger than 1,000 mW. For example, to activate the first set of UV-C light emitting diodes of the light source optionally comprises to emit UV-C light with a power in the range from 100 mW to 900 mW, 200 mW to 800 mW, 300 mW to 700 mW, and/or 350 mW to 600 mW. For example, to activate the first set of UV-C light emitting diodes of the light source optionally comprises to emit UV-C light with a power of 250 mW, 300 mW, 350mW 400 mW, 450 mW, 500 mW, and/or 550 mW.

In one or more exemplary decontamination devices, to activate a second set of light emitting diodes of the light source comprises to emit UV-A light with a power larger than 200 mW, e.g. in the range from 500 mW to 5 W. To activate a second set of light emitting diodes of the light source comprises to emit UV-A light with a power in the range from 1 mW to 200 mW or in the range from 200 mW to 500 mW for each second LED. In one or more exemplary decontamination devices, to activate a second set of light emitting diodes of the light source comprises to emit UV-A light with a power in the range from 500 mW to 2 W, such as in the range from 1W to 1.7W, e.g. in the range from 1.2 W to 1.4 W, optionally at 700 mA, or even larger than 2 W for each UV-A LED of the second set of UV-A light emitting diodes.

The decontamination device comprises a housing. The housing may be an elongated housing. The decontamination may have a first end and a second end with a center axis extending between the first end and the second end. The housing comprises a head part and a handle part.

The head part optionally comprises the UV-transparent area. In other words, the UV-transparent area may be arranged in the head part and light from the light source optionally exits the decontamination device though a UV-transparent area of the head part. The head part may be arranged at or form the first end of the housing/decontamination device. The UV-transparent area may be arranged in a first end surface of the first end. The UV-transparent area may be arranged in a side surface of the housing.

The head part may have a substantially frustoconical shape. The decontamination device/housing may substantially have the shape of a torch and/or a flashlight, e.g. formed by the head part and/or the handle part. The head part may comprise a heat sink e.g. a first heat sink, such as a cooling head providing a heat sink for the decontamination device. The head part may comprise a body part and an outer part, such as an outer ring arranged around the body part. The head part may comprise a coupling device. The coupling device may comprise a coupling element, such as a ring-shaped coupling element. The coupling device, such as the coupling element, may be arranged at the first end of the housing. The head part may have a radius (perpendicular to the central axis) in the range from 1 cm to 5 cm, e.g. from 3 cm to 4 cm.

In one or more example decontamination devices, the head part comprises a context ring. The context ring may provide a protection ring for the head part and/or may be used to indicate a brand name on it. The context ring may be used to indicate security instructions for the user of the decontamination device.

The handle part may be substantially cylindrical in shape. The handle part may comprise one or a plurality of handle parts, e.g. including a first handle part and/or a second handle part. The handle part have a length in the range from 3 cm to 30 cm, such as in the range from 5 to 15 cm. The handle part may have a radius in the range from 1 cm to 5 cm, e.g. from 2 cm to 3 cm. The handle part may be configured for a user to hold when operating the decontamination device, such as when a user uses the decontamination device to decontaminate a surface, e.g. the surface of an object or a component. The handle part may comprise a gripping portion, such as a gripping layer, such that a comfortable and secure grip may be provided for the user. The gripping portion may comprise a material providing increased friction between the gripping portion and the hand of the user, such as a rubber material e.g. silicone. The gripping portion may comprise a surface comprising structures for providing a rougher surface with more surface area compared to a smooth surface. The gripping portion may provide an increased grip for safer handling of the decontamination device.

The handle part may comprise a first handle part, a second handle part, and optionally a third handle part.

The decontamination device may comprise a power source cavity for accommodating or fitting a power source, such as one or more batteries and/or battery packs. The decontamination device may comprise one or more batteries e.g. a battery pack comprising one or more batteries, such as one battery, two batteries, three batteries. The one or more batteries may comprise one or more of Lithium ion batteries, Nickel ion batteries, and/or Lead ion batteries. The power source may be rechargeable, e.g. via second interface. The power source may include a fuel cell and/or a solar cell.

The decontamination device may be configured for attachment to accessory equipment, such as a carrier cord or a carrier handle. Thus, the decontamination device may comprise, e.g. as part of the handle part a connector or attachment ring.

The decontamination device may comprise a second interface. The second interface may comprise a charging interface and/or a configuration interface, such as a connector for charging and/or configuring the decontamination device. The decontamination device, such as the second interface, may comprise an indicator, e.g. for indicating a status of the decontamination device, such as a light indicator. The indicator may be arranged at the second end of the housing. For example, the light indicator may indicate one or more of the following statuses: on, off, active, inactive, operation state, battery status, transmitting, and/or receiving. The decontamination device may comprise an end cap, e.g. for providing a tight closing of the second interface, such as a plug. In some embodiments, the end cap may be substantially transparent, such as light transparent, whereby the indicator of the second interface may be visible to the user when the end cap is on. The decontamination device may comprise a protection ring, e.g. at the second end of the housing. The protection ring may for example be a metal protection ring for protecting the decontamination device e.g. against impacts such as when the decontamination device is dropped.

The decontamination device, such as the head part, optionally comprises a heat sink for cooling the light source. The light source optionally comprises a circuit board. The circuit board may be made of material comprising a first layer of first material with a first thermal conductivity and a first heat capacity. The light source optionally comprises a first set of UV-C light emitting diodes mounted on the circuit board. The heat sink may be in thermal contact with the first layer and made of a second material with a second thermal conductivity and a second heat capacity. The second heat capacity is optionally larger than the first heat capacity.

In one or more example decontamination devices, the first thermal conductivity is larger than the second thermal conductivity. In one or more example decontamination devices, the first material comprises copper. In one or more example decontamination devices, the second material is aluminium. In one or more example decontamination devices, the heat sink may form an outer surface of the head part. In one or more example decontamination devices, the heat sink comprises a coating on the outer surface. The coating may have a thickness less than 300 μm.

In one or more example decontamination devices, the heat sink may comprise a plurality of channels extending parallel to a longitudinal axis of the decontamination device. In other words, the heat sink may comprise one or more cooling elements, e.g. one or more cooling passages arranged circumferentially to the head part, such as between the body part and the outer part. In other words, the heat sink may comprise one or more channels, such as cooling ducts, providing a cooling effect for the decontamination device, e.g. by transporting the heat generated by the decontamination device, such as the light source, away from the decontamination device. The heat sink may therefore use convection and/or radiation for transferring/transporting the heat away from the decontamination device. For example, the heat sink may use a chimney effect of the one or more channels to transport the heat away from the light source, e.g. by providing a “hot side” and a “cold side” in the one or more channels for creating a flow of air, e.g. in the one or more channels. The one or more channels may be arranged between the body part and the outer part may, such that the body part and the outer part may be in contact, e.g. forming walls between the one or more channels. The heat sink, such as the outer surface of the head part and/or the one or more channels may be in direct contact with air. The decontamination device may thereby provide forced convection, e.g. in the head part.

A decontamination assembly is disclosed, wherein the decontamination assembly comprises a UV shield and a decontamination device, e.g. a decontamination device as disclosed herein. The decontamination device optionally comprises a coupling device for coupling, e.g. releasably coupling, the decontamination device/head part to a port, e.g. of the UV shield. The coupling device may be arranged in the head part, e.g. at the first end of the housing. In other words, the head part may comprise the coupling device. In one or more exemplary decontamination devices, the coupling device is a separate coupling element, e.g. attached to the heat sink. The coupling device may be integrated in the heat sink.

The UV shield provides an increased safety for the user and allows a user to use different UV shields for different decontamination procedures.

Also, a decontamination system is disclosed, the decontamination system comprising a container having an inner volume defined by at least one wall including a first wall, and a carrier device rotatably arranged about a rotation axis in the inner volume. The decontamination system optionally comprises a motor configured to rotate the carrier device. The decontamination system, e.g. the container, may comprise a first port in the first wall of the container, wherein the first port is configured to couple a decontamination device to the container. The decontamination system, e.g. the container, may comprise a second port in a wall of the container, wherein the second port is configured to couple a decontamination device to the container. It is to be understood that description of a port of a UV-shield is also applicable to a port of the decontamination system and vice versa.

The coupling device of the decontamination device may be configured for releasably coupling the decontamination device to a port of the container. The coupling device of the decontamination device may comprise a coupling element. The coupling element may be ring-shaped optionally encircling the UV-transparent area. In other words, the coupling element may have an opening forming the UV-transparent area. The coupling element may be a coupling ring. The coupling ring may be attached to the heat sink. The coupling device, such as the coupling element, of the decontamination device may comprise a plurality of rods or protrusions extending radially, e.g. radially outwards or radially inwards. The plurality of rods/protrusions may be evenly distributed along an outer periphery of the coupling device/coupling element/coupling ring. The plurality of protrusions may comprise at least three protrusions, such as three, four, five, six, seven, eight or more protrusions.

The coupling device may comprise a plurality of recesses, e.g. in the outer surface of the head part, such as in a radially outward facing surface of the coupling device/coupling element. The plurality of recesses may extend in the inner surface of the coupling device/coupling ring, such as in a radially inward facing surface of the coupling device/coupling element.

The plurality of recesses may be evenly distributed along an outer periphery of the coupling device/coupling element/coupling ring. The plurality of recesses may comprise at least three recesses, such as three, four, five, six, seven, eight or more recesses. The coupling device may comprise an inner threading or an outer threading, e.g. on a radially inward or radially outward facing surface of the head part/coupling element, such as on one or more protrusions. The coupling element may be configured for locked attachment to the heat sink of the head part, e.g. by means of locking protrusions, e.g. evenly distributed along an outer periphery of the coupling element/coupling ring. The coupling device may comprise a bayonet coupling part for coupling to a corresponding bayonet coupling part of the UV shield. The plurality of recesses and/or protrusions may form the bayonet coupling part.

In one or more example decontamination assemblies and/or decontamination devices, the UV shield is made of a UV absorbing polymer material. For example, the UV shield may be made of polycarbonate, polypropylene, an acrylic, polyethylene, ABS, POM, PEEK, or DelRin. The UV shield may be made of metal or coated with metal or metal alloy on inner and/or outer surface. The UV shield may be coloured. For example, the UV shield may be orange, red, cyan, green, or blue. Coloured UV-shields allow for visual inspection of fluorescence from the item or organic material on the item to be decontaminated. The inner surface and/or the outer surface of the UV-shield may comprise an optical high pass filter and/or an optical low pass-filter. The optical high pass filter and/or the optical low pass-filter may be a coating, e.g. vacuum-deposited on body of UV-shield. The UV-shield, e.g. one or more of the wall(s) defining the inner volume may be transparent or opaque thereby allowing visual inspection of the item during decontamination. In other words, the UV-shield may be configured for visual inspection of the inner volume through the wall(s) of the UV shield. In one or more example decontamination assemblies and/or decontamination devices, the UV shield has an inner volume and comprises a rim defining an opening for accessing the inner volume. The opening of the UV shield allows for arranging an item within the inner volume, e.g. by the rim contacting a surface on which the item is placed. Thereby, the UV shield shields a user from UV light from the light source during decontamination of the item. In one or more example decontamination assemblies and/or decontamination devices, the opening has an area of in the range from 25 cm² to 500 cm². The opening may have a largest extension in the range from 5 cm to 50 cm. The opening may have a smallest extension in the range from 1 cm to 30 cm.

In one or more example decontamination assemblies and/or decontamination devices, the rim is circular or polygonal. In other words, the opening may be circular, oval, polygonal or other shape which facilitates arranging an item within the inner volume of the UV shield. The opening may be rectangular optionally with rounded corners. The UV shield has an inner volume for at least partly accommodating an item to be decontaminated. The inner volume may have a volume in the range from 50 cm³ to 5,000 cm³. The UV shield comprises one or more walls defining the inner volume. The UV shield may comprise a first part and optionally a second part. The first part may be conical. The second part may be cylindrical. The UV shield may extend from a first end to a second end along a center axis. The port of the UV shield may be arranged at the first end. The opening/rim of the UV shield may be arranged at the second end of the UV shield. The first part may extend from the first end towards the second end. The second part may extend from the second end towards the first end. A first inner surface of the first part may partly define the inner volume and optionally has an increasing cross-sectional area towards the second end, e.g. along the center axis. The first part/first inner surface may be conical. A second inner surface of the second part may partly define the inner volume and optionally has a constant, decreasing, or increasing cross-sectional area towards the second end, e.g. along the center axis. The second part/second inner surface may be cylindrical, e.g. have a constant and circular, oval, polygonal cross-sectional area, e.g. along the center axis. The first part may have a length (extension along center axis) in the range from 1 cm to 10 cm. The second part may have a length (extension along center axis) in the range from 1 cm to 20 cm. In one or more exemplary UV shields, the second part has a length in the range from 2 cm to 5 cm or from 5 cm to 10 cm.

In one or more example decontamination assemblies and/or decontamination devices, the UV shield, or at least a first part of the UV shield is conical or at least partly conical.

The UV shield has a port, also denoted first port, for releasably coupling the decontamination device and the UV shield. The UV-shield may comprise a plurality of ports including a first port and a second port. Thereby, multiple decontamination devices may be coupled to the UV-shield and/or a decontamination device can be used in different coupling arrangements depending on the item to be decontaminated which in turn increases the user flexibility.

A port may have an opening for passage of UV light from the decontamination device into the inner volume. For example, the (first) port of the UV shield may have a (first) opening with an area in the range from 10 cm² to 50 cm². The (first) opening of the (first) port may be circular or substantially circular. The port may comprise a coupling structure, such as a bayonet coupling device, for releasably coupling the port/UV shield to the decontamination device. The coupling structure may comprise one or more recesses and/or protrusions, optionally extending radially from a center of the opening, in an edge forming and/or defining the opening.

The decontamination system comprises a container with one or more ports, such as a first port and/or a second port. In one or more exemplary decontamination systems, the first port and/or the second port comprises a bayonet coupling device for releasably coupling a decontamination device to the container. In one or more exemplary decontamination systems, the first port comprises a first closure element for closing a first opening of the first port. The second port may comprise a second closure element for closing a second opening of the second port. In one or more exemplary decontamination systems, the container is made of a UV absorbing polymer material. For example, the container may be made of polycarbonate, polypropylene, an acrylic, polyethylene, ABS, POM, PEEK, or DelRin. The container may be made of metal or coated with metal or metal alloy on inner and/or outer surface. The surface of the container may comprise high pass filter and/or a low pass-filter. The material of the container and/or the UV shield may be transparent or at least partly transparent for visible light. Thereby, a user can follow the decontamination procedure during decontamination or the item/surface. In one or more exemplary decontamination systems, the first port is configured to attach a decontamination device such that a first beam from the decontamination device coupled to the first port is in a first direction. The second port may be configured to attach a decontamination device such that a second beam from the decontamination device coupled to the second port is in a second direction. The first direction and the second direction may form a beam angle larger than 30°, such as in the range from 45° to 180°. The beam angle may be in the range from 60° to 120°. Thereby an improved exposure of the item to be decontaminated may be provided in the decontamination system. In one or more exemplary decontamination systems, a first angle between the rotation axis and the first direction is less than 60°, such as less than 30°. The first angle between the rotation axis and the first direction may be in the range from 15° to 60°. The first angle may, e.g. in a single-port container, be in the range from 60° to 90°, such as in the range from 75° to 90°, e.g. about 90°. In one or more exemplary decontamination systems, a second angle between the rotation axis and the second direction is larger than 30°. The second angle between the rotation axis and the second direction may be in the range from 15° to 90°, such as in the range from 30° to 60°. In one or more exemplary decontamination systems, the container comprises a rotation opening, and wherein the carrier device comprises a shaft rotatably arranged in the rotation opening. The shaft may be a telescopic arm. A rotation opening with a rotatably arranged shaft therein, allows rotation of the carrier device by a motor arranged outside the container.

In one or more exemplary decontamination systems, the carrier device comprises a frame part and a seat part, wherein the seat part comprises a seat configured for accommodating an item to be decontaminated, wherein the seat part is made of a seat material made of a UV transparent material, such as silicone. The item may be press-fitted in the seat, and UV transparent material may allow for a 360° exposure of the item during rotation in turn allowing a more efficient decontamination procedure, e.g. by eliminating the need for a double-position decontamination procedure where the item is first decontaminated in a first position on the carrier device and then rearranged and then secondly decontaminated in a second position on the carrier device. In one or more exemplary decontamination systems, the decontamination system comprises a first decontamination device and optionally a second decontamination, wherein the first decontamination device is configured to releasably couple to the first port and/or the second port, and optionally the second decontamination device is configured to releasably couple to the second port. The first decontamination device and/or the second decontamination device may be a decontamination device as disclosed herein.

The decontamination device comprises a light source configured to emit UV-C light. The light source may be configured to be arranged or housed in the head part. The light source may be configured to emit UV-C light with a power in the range from 1 mW to 20 mW or in the range from 20 mW to 50 mW. In one or more exemplary decontamination devices, the light source is configured to emit UV-C light with a power larger than 10 mW, e.g. in the range from 20 mW to 400 mW. The light source may be configured to emit UV-C light with a power in the range from 50 mW to 1,000 mW or even larger than 1,000 mW. For example, the light source may be configured to emit UV-C light with a power in the range from 100 mW to 900 mW, 200 mW to 800 mW, 300 mW to 700 mW, and/or 350 mW to 600 mW. For example, the light source may be configured to emit UV-C light with a power of 250 mW, 300 mW, 350mW 400 mW, 450 mW, 500 mW, and/or 550 mW. By UV-C light it may be understood that the light source may be configured to emit light having a wavelength in the range of 200 nm to 280 nm, such as in the range from 255 to 275 nm, e.g. light with a peak wavelength about 265 nm.

In one or more exemplary decontamination devices, the decontamination device is configured to emit UV-C light for a time period less than 2 minutes, e.g. less than 1 minute, such as less than 30 seconds or even less than 20 seconds, e.g. upon activation of the decontamination device via the interface.

The light source may comprise a circuit board. The heat sink may be in thermal contact with the first layer and made of a second material with a second thermal conductivity and a second heat capacity, wherein the second heat capacity is larger than the first heat capacity. In one or more example decontamination devices, the circuit board may act as a heat sink, e.g. a second heat sink. The heat sink and the circuit board, in other words the first heat sink and the second heat sink may be in thermal contact. In other words, the circuit board may use conduction for transferring the heat from the light source stored in the circuit board to the heat sink. The decontamination device may provide one or more of conduction, convection, and radiation heat transfer for cooling the light source.

In one or more example decontamination devices, the circuit board comprises one or more cut-outs, such as a first set of cut-outs. The one or more cut-outs may be through openings through the circuit board, such that the heat may not be conducted passed the cut-out. The one or more cut-outs may be provided around a light emitting diode, such as a UV light emitting diode, UV LED. The one or more cut-outs may substantially insulate thermally each UV LED from each other, and/or substantially insulate thermally a light emitting diode from a UV LED. Further, the one or more cut-outs may substantially direct the heat conduction from the circuit board to the heat sink. The one or more cut-outs may be substantially V-shaped and/or arc-shaped. The one or more cut-outs may be arranged such that the open part of the V is oriented in direction of the heat sink, e.g. away from the center of the circuit board.

In one or more example decontamination devices, the light source may comprise a first set of UV-C light emitting diodes, LEDs, mounted on the circuit board. The first set of UV-C light emitting diodes may comprise one or more UV-C light emitting diodes, such as a plurality of UV-C light emitting diodes. In one or more example decontamination devices, the first set of UV-C light emitting diodes may comprise between three and twenty UV-C light emitting diodes, such as in the range from four to ten UV-C light emitting diodes. Thereby increased design freedom and control of shape and/or distribution of UV-C light is provided. For example, the first set of UV-C light emitting diodes may comprise a first primary LED, optionally a first secondary LED, and optionally a first tertiary LED. The first set of UV-C light emitting diodes may comprise a first quaternary LED and/or a first quinary LED. The first set of UV-C light emitting diodes may comprise six or more LEDs including a first senary LED optionally arranged as a center diode in a diode configuration. In other words, the light source may comprise a first set of light elements comprising a first primary light element, such as a first primary LED, a first secondary light element, such as a first secondary LED, and a first tertiary light element, such as a first tertiary LED, mounted on a proximal surface of the circuit board. The first set of light elements may comprise a first set of UV-C light emitting diodes, LEDs, mounted on the circuit board. For example, the first set of UV-C light emitting diodes may comprise a first primary LED, a first secondary LED, and optionally a first tertiary LED. The first set of UV-C light emitting diodes may comprise a first quaternary LED optionally arranged as a center diode in a diode configuration, e.g. where the other first LEDs of the first set of LEDs are arranged around the first quaternary LED. In one or more example decontamination devices, a first primary LED of the first set of UV-C light emitting diodes may be arranged as a center diode in a diode configuration, e.g. where a second set of LEDs are arranged around the first primary LED.

In one or more example decontamination devices, the LEDs of the first set of UV-C light emitting diodes are configured to emit UV-C light in the range from 260 nm to 270 nm, e.g. with a peak wavelength in the range from 260nm to 270 nm, e.g. about 265 nm. A light source with UV-C light emitting diodes is advantageous due to the instant provision of high or maximum effect at turn-on and lack of heat-up time. Further, UV-C light emitting diodes can sustain to be turned on/off up to more than 100,000 times. One or more UV-C LEDs of the first set of UV-C light emitting diodes may have an optical power output, i.e. configured to emit UV-C light with a power, in the range from 1 mW to 20 mW or in the range from 20 mW to 50 mW. In one or more exemplary decontamination devices, the UV-C LEDs of the first set of UV-C light emitting diodes may have an optical power output light source in the range from 50 mW to 150 mW or even larger than 150 mW. For example, the UV-C LEDs of the first set of UV-C light emitting diodes may each have an optical power output light source in the range from 65 mW to 95 mW, e.g. at 500 mA.

In one or more example decontamination devices, the light source may comprise a second set of light emitting diodes mounted on the circuit board. The LEDs of the second set of light emitting diodes are optionally configured to emit visible light and/or UV light comprising UV-A light. The light source, such as the second set of light emitting diodes, may be configured to emit visible light e.g. having a wavelength in the range of 380 nm to 740 nm. The light source, such as the second set of light emitting diodes, may be configured to emit UV light comprising UV-A light e.g. having a (peak) wavelength in the range from 320 nm to 380 nm. The light source, such as the second set of light emitting diodes, may be configured to emit UV-A light, e.g. light in the range from 360 nm to 400 nm. For example, the upper limit of the range of UV-A light, such as 380 nm, may be visible to the human eye.

In one or more example decontamination devices, the second set of light emitting diodes comprises one or a plurality of UV-A light emitting diodes. For example, the second set of light emitting diodes may be UV-A light emitting diodes and optionally comprises one or a plurality of LEDs, such as two, three, four, five, six or more LEDs. The second set of light emitting diodes may comprise a second primary LED, optionally a second secondary LED, and optionally a second tertiary LED. The second set of UV-A light emitting diodes may comprise a second quaternary LED and/or a second quinary LED. The second set of UV-A light emitting diodes may comprise six or more LEDs including a second senary LED. A single second LED may constitute the second set of light emitting diodes, i.e. the second set of LEDs may comprise one LED, such as one UV-A LED. The second set of UV-A light emitting diodes may comprise a second quaternary LED, e.g. arranged as a center diode in a diode configuration. The second primary LED may be arranged as a center diode, e.g. where the first set of LEDs are arranged around the second primary LED.

One or more UV-A LEDs of the second set of light emitting diodes may have an optical power output, i.e. configured to emit UV-A light with a power, in the range from 1 mW to 200 mW or in the range from 200 mW to 500 mW. In one or more exemplary decontamination devices, the UV-A LEDs of the second set of UV-A light emitting diodes may have an optical power output light source in the range from 500 mW to 2 W or even larger than 2 W. For example, the UV-A LEDs of the second set of UV-A light emitting diodes may have an optical power output light source in the range from 1W to 1.7W, e.g. in the range from 1.2 W to 1.4 W, optionally at 700 mA.

In one or more example decontamination devices, the light source comprises a third light emitting diode configured to emit visible light. The third light emitting diode may be configured to emit blue light. A light source emitting a combination of UV-C light and visible light is advantageous in that the visible light may indicate to the user, which parts of a surface or item that are exposed to UV-C light, which in turn provides a decontamination device that is safe to use.

In one or more example decontamination devices, UV-C light emitting diodes (first set) and/or the UV_A light emitting diodes (second set) are distributed around a center diode, such as a UV-C light emitting diode, a UV-A light emitting diode, or the third light emitting diode, positioned in a center part of the circuit board. In other words, first set of UV-C light emitting diodes may be distributed, e.g. evenly distributed, around a center diode, such as the third light emitting diode. Thus, the third light emitting diode may be a center diode. The second set of UV-A light emitting diodes may be distributed, e.g. evenly distributed, around a center diode, such as the third light emitting diode. First LEDs and second LEDs may be alternately arranged around the center diode, such as a UV-C light emitting diode, a UV-A light emitting diode, or the third light emitting diode. In other words, second LEDs may be respectively arranged between two first LEDs and/or first LEDs may be respectively arranged between second LEDs. It is noted that a UV-C LED, such as first quaternary LED, may be used as the center diode. In other words, a first LED, e.g. first quaternary LED or first senary LED, of the first set of LEDs may be a center diode, optionally with the remaining first LEDs of the first set of LEDs arranged around the center diode. It is noted that a UV-A LED, such as second quaternary LED, may be used as the center diode. In other words, a second LED, e.g. second quaternary LED of the second set of LEDs may be a center diode, optionally with the remaining second LEDs of the second set of LEDs arranged around the center diode.

In one or more example decontamination devices, the light source has in the range from five to ten light emitting diodes mounted on the circuit board. For example, the light source may comprise a first set of LEDs with from two to six UV-C LEDs and a second set of LEDs with from two to four UV-A LEDs.

In one or more example decontamination devices/light sources, one or more center-to-center distances between two neighbouring light emitting diodes is in the range from 10 mm to 25 mm. In one or more exemplary light sources, a minimum distance between neighbouring light emitting diodes is larger than 5 mm or larger than 10 mm. In one or more exemplary light sources, a minimum distance between neighbouring light emitting diodes is less than 35 mm, such as about 18 mm.

In one or more example decontamination devices, the first set of UV-C light emitting diodes are mounted in a first height on the circuit board and the third light emitting diode is mounted in a third height on the circuit board, wherein the third height is different from the first height, such as larger than the first height. In one or more exemplary light sources, the difference between the third height and the first height is larger than 10 my, such as in the range from 10 to 100 my. Different heights for the first set of LEDs and the third LED arranges the first LEDs and the third LED at different distances from the first end of the housing and/or in different positions along the central axis in relation to their respective reflector cavities, which in turn allows control of the area exposed by the first set of LEDs and the third LED, e.g. such that a first area exposed/illuminated/irradiated with light from the first set of LEDs is smaller than a third area exposed/illuminated/irradiated with light from the third LED. Thereby is ensured that the user can see the UV-C exposed area and therefore avoid exposing e.g. the user or other persons to UV-C radiation from the decontamination device.

In one or more example decontamination devices, the light source comprises a spacer arranged between the circuit board and the third light emitting diode. Thereby a precise positioning of the third light emitting diode is provided, which in turn allows improved control of the height difference between first set of LEDs and/or the second set of LEDs and the third LED.

In one or more example decontamination devices, the first set of UV-C light emitting diodes or at least a part thereof are mounted on the circuit board with a first radial distance from a center of the circuit board, e.g. from the center diode, such as a third light emitting diode. The first radial distance may be in the range from 10 mm to 40 mm, such as in the range from 15 mm to 30 mm, e.g. about 18 mm. In one or more example decontamination devices where the first set of UV-C light emitting diodes comprises M LEDs, M−1 first LEDs of the first set of UV-C light emitting diodes are mounted on the circuit board with a first radial distance from a center of the circuit board, e.g. from the center diode being the M′th first LED of the first set of UV-C light emitting diodes. M may be three, four, five, six, seven or even larger than seven.

In one or more example decontamination devices, the second set of light emitting diodes are mounted on the circuit board with a second radial distance from a center of the circuit board, e.g. from the center diode, such as a first light emitting diode or a third light emitting diode. The second radial distance may be in the range from 10 mm to 40 mm, such as in the range from 15 mm to 30 mm, e.g. about 18 mm. The first radial distance may be different from the second radial distance. In one or more example decontamination devices where the second set of light emitting diodes comprises N LEDs, N−1 second LEDs of the second set of light emitting diodes are mounted on the circuit board with the first or the second radial distance from a center of the circuit board, e.g. from the center diode being the N′th second LED of the second set of light emitting diodes. N may be three, four, five, six, seven or even larger than seven.

The decontamination device comprises a light guide for guiding light, such as UV light comprising UV-C and/or UV-A light, from the light source towards a UV-transparent area, e.g. in the head part of the housing. The head part/UV-transparent area may comprise a window part in the head part, e.g. arranged at or within 30 mm from the first end of the housing/head part. The UV-transparent area may comprise a disc or sheet made of a substantially UV-transparent material, such as a borosilicate glass or a silicone glass. The disc or sheet may be a covering window or disc, e.g. for protecting the elements inside the head part of the decontamination device.

The light guide may comprise a reflector device, such as a UV reflector device, having a reflector structure in a proximal surface of the reflector device. The reflector structure may comprise one or more, such as a plurality of reflector cavities. The reflector cavities may comprise a first set of reflector cavities and optionally a second set of reflector cavities. The number of reflector cavities in the reflector structure may correspond to or be larger than the number of LEDs of the light source.

The first set of reflector cavities comprises one or more first reflector cavities including a first primary reflector cavity having a first primary reflector surface, and optionally a first secondary reflector cavity having a first secondary reflector surface. The first set of reflector cavities may include a first tertiary reflector cavity having a first tertiary reflector surface, and optionally a first quaternary reflector cavity having a first quaternary reflector surface. First reflector cavities may be aligned with respective first light emitting diodes of the light source. Accordingly, the number of reflector cavities in the first set of reflector cavities may be the same as the number of LEDs in the first set of LEDs. For example where the light source comprises four first LEDs of first set of LEDs, the reflector device may comprise four first reflector cavities. The first quaternary reflector cavity or a first senary reflector cavity may be arranged as a center reflector cavity, e.g. with other reflector cavities, such as remaining first reflector cavities of the first set of reflector cavities and/or second reflector cavities of the second set of reflector cavities arranged around the center reflector cavity.

The second set of reflector cavities comprises one or more second reflector cavities including a second primary reflector cavity having a second primary reflector surface, and optionally a second secondary reflector cavity having a second secondary reflector surface. The second set of reflector cavities may include a second tertiary reflector cavity having a second tertiary reflector surface, and optionally a second quaternary reflector cavity having a second quaternary reflector surface. Second reflector cavities may be aligned with respective second light emitting diodes of the light source. Accordingly, the number of reflector cavities in the second set of reflector cavities may be the same as the number of LEDs in the second set of LEDs.

A reflector cavity has a reflector surface. The reflector surfaces of the reflector cavities or at least part thereof may be metallic surfaces.

In one or more exemplary light guides, the reflector surfaces of the reflector cavities or at least the first reflector surfaces are made of or at least comprises PTFE, such as e-PTFE (expanded PolyTetraFluor Ethylen).

In one or more exemplary decontamination devices, first reflector surfaces, such as the first primary reflector surface and/or the first secondary reflector surface are metallic surfaces, such as aluminium, gold, copper, silver, rhodium, or platinum. The first tertiary reflector surface may be a metallic surface.

In one or more exemplary decontamination devices, second reflector surface(s), such as the second primary reflector surface and/or the second secondary reflector surface are metallic surfaces. The second tertiary reflector surface may be a metallic surface.

The light guide/UV reflector device may be configured to reflect and/or direct the light emitted from the light source towards a UV-transparent area in the head part of the housing for irradiating a surface to be decontaminated by the decontamination device.

The UV reflector device may comprise a reflector cavity for each LED in the light source. In other words, the number of reflector cavities may correspond to the number of light emitting diodes or elements of the light source.

In one or more example decontamination devices, the light guide, such as the reflector device, may comprise one or more protrusions and/or recesses in a distal surface of the reflector device. For example, the reflector device optionally comprises one or more protrusions, such as two protrusions, three protrusions, and/or four protrusions extending from the distal surface of the reflector device. The protrusion(s) may be alignment protrusions for aligning the light guide and the light source. In an assembled state of the decontamination device, the one or more alignment protrusions in the distal surface of the reflector device may be at least partly accommodated in one or more alignment openings of the light source and optionally in the spacer plate and/or the spacer ring. In other words, the reflector device, the circuit board, the spacer plate, and/or the spacer ring may form an assembly that is substantially connected to each other, when the one or more alignment protrusions of the reflector device are accommodated in the one or more alignment openings. Thereby, the LEDs of the light source and the reflector cavities may be aligned to precisely arrange the LEDs in relation to the reflector cavities, thereby improving intensity of the UV-light emitted through the UV-transparent area.

In one or more example decontamination devices, the reflector device comprises a reflector body and a first layer coated onto the reflector body, wherein the reflector body is made of a body material and the first layer is made of a first reflector material. The first reflector material may be metallic. The first reflector material may comprise a metal being one or more of aluminium, gold, copper, silver, rhodium and platinum. The first reflector material may comprise a metal alloy comprising one or more of aluminium, gold, copper, silver, rhodium and platinum. The first reflector material may comprise PTFE, such as e-PTFE (expanded PolyTetraFluor Ethylen) or microporous PolyTetraFluor Ethylen. The reflector device may comprise a second layer coated onto the first layer of the reflector device. The first layer and/or the second layer may form the reflector surfaces of the reflector cavities.

In one or more example decontamination devices, the reflector surfaces, such as the first reflector surfaces of the first set of reflector cavities and/or the second reflector surface(s) of the second set of reflector cavities, are concave surfaces, such as concave aspheric surfaces. In other words, a cross section of a reflector cavity may be concave aspheric in shape. The reflector structure may therefore act as an aspherical concave optic, such as lens, in the decontamination device. For example, the plurality of reflector cavities may be overlapping, such as overlapping cones, to provide the aspherical concave shape of the reflector structure. The reflector device may be configured to reflect at least 50% of the light emitted from the light source, at least 60% of the light emitted from the light source, at least 70% of the light emitted from the light source, at least 80% of the light emitted from the light source, at least 90% of the light emitted from the light source, and/or at least 93% of the light emitted from the light source.

The reflector device may comprise respective openings, such as a first set of (reflector) openings and/or a second set of (reflector) openings, in the respective reflector surfaces, such as first openings in respective first reflector cavities and/or second openings in respective second reflector cavities, e.g. for forming light passages between the reflector cavities and a distal side of the reflector device and/or to allow positioning respective LEDs in or at respective reflector cavities for provision of light to be reflected by the reflector surfaces. First reflector openings may be circular, e.g. with a diameter in the range from 2 mm to 15 mm, such as between 8 mm and 10 mm. Second reflector openings may be circular, e.g. with a diameter in the range from 2 mm to 15 mm, such as between 8 mm and 10 mm.

In one or more example decontamination devices, the reflector device comprises a first primary reflector opening in the first primary reflector surface, e.g. for forming a first primary light passage between the first primary reflector cavity and a distal side of the reflector device and/or to allow positioning a first primary LED in or at the first primary reflector cavity for provision of light to be reflected by the first primary reflector surface. The first primary reflector opening may be circular, e.g. with a diameter in the range from 2 mm to 15 mm, such as between 8 mm and 10 mm. The reflector device may comprise a first secondary reflector opening in the first secondary reflector surface, e.g. for forming a first secondary light passage between the first secondary reflector cavity and a distal side of the reflector device and/or to allow positioning a first secondary LED in or at the first secondary reflector cavity for provision of light to be reflected by the first secondary reflector surface. The first secondary reflector opening may be circular, e.g. with a diameter in the range from 2 mm to 15 mm, such as between 8 mm and 10 mm.

The reflector device may comprise a plurality of reflector openings, e.g. a reflector opening for each reflector cavity. In other words, the reflector device may comprise a number of reflector openings corresponding to the number of LEDs of the light source, where each LED is arranged at or in a respective reflector opening for emitting light via the respective reflector cavity associated therewith. The reflector structure may comprise four, five, six, seven or more reflector openings. The reflector openings may be circular, e.g. with a diameter in the range from 2 mm to 15 mm, such as between 8 mm and 10 mm.

The reflector device may comprise a second primary reflector opening in the second primary reflector surface, e.g. for forming a second primary light passage between the second primary reflector cavity and a distal side of the reflector device and/or to allow positioning a second primary LED in or at the second primary reflector cavity for provision of light to be reflected by the second primary reflector surface. The second primary reflector opening may be circular, e.g. with a diameter in the range from 2 mm to 15 mm, such as between 8 mm and 10 mm.

In one or more example decontamination devices, the reflector structure comprises four, five, six, seven or more reflector cavities. In a reflector structure with at least four reflector cavities, at least three, such as four, five, six, or more reflector cavities including the first primary reflector cavity, the first secondary reflector cavity, and the second primary reflector cavity, may be distributed around a center reflector cavity also denoted first quaternary reflector cavity, second quaternary reflector cavity, or third reflector cavity.

In one or more example decontamination devices, the first set of reflector cavities and optionally the second set of reflector cavities are, e.g. evenly, distributed around a third reflector cavity.

The reflector structure may comprise one or more of a first tertiary reflector cavity of the first set of reflector cavities, a second secondary reflector cavity and a second tertiary reflector cavity of the second set of reflector cavities, and/or a third reflector cavity. In one or more exemplary light guides, the reflector structure comprises a first quaternary reflector cavity e.g. arranged as a center reflector cavity.

The first tertiary reflector cavity has a first tertiary reflector surface, the second secondary reflector cavity has a second secondary reflector surface, the second tertiary reflector cavity has a second tertiary reflector surface, and the third reflector cavity (if present) optionally has a third reflector surface.

The reflector device may comprise a first tertiary reflector opening in the first tertiary reflector surface, wherein the first tertiary reflector opening is optionally circular with a diameter in the range from 2 mm to 15 mm. The reflector device may comprise a second secondary reflector opening in the second secondary reflector surface, wherein the second secondary reflector opening is optionally circular with a diameter in the range from 2 mm to 15 mm. The reflector device may comprise a second tertiary reflector opening in the second tertiary reflector surface, wherein the sixth reflector opening is optionally circular with a diameter in the range from 2 mm to 15 mm. The reflector device may comprise a third reflector opening in the third reflector, wherein the third reflector opening is optionally circular with a diameter in the range from 2 mm to 15 mm.

In one or more example decontamination devices, a center-to-center distance between two neighbouring reflector cavities is in the range from 10 mm to 25 mm, such as in the range from 15 mm to 20 mm, e.g. about 18 mm.

In one or more example decontamination devices, the light source is configured to emit visible light and/or UV light comprising UV-A light. In one or more example decontamination devices, the light source may comprise one or more UV-A light emitting diodes. UV-A LEDs may be preferred, e.g. as second light emitting diodes due to their combined UV-A and visible component. In other words, the second set of light emitting diodes may be UV-A diodes.

In one or more example decontamination devices, the reflector device comprises a plurality of damping recesses including a first (damping) recess and a second (damping) recess in the proximal surface of the reflector body. In one or more example decontamination devices, the decontamination device comprises a first damping element at least partly accommodated in the first recess, and a second damping element at least partly accommodated in the second recess. The reflector device optionally comprises a third (damping) recess in the proximal surface of the reflector body. In one or more example decontamination devices, the decontamination device comprises a third damping element at least partly accommodated in the third recess. The first damping element, the second damping element, and/or the third damping element may for example comprise one or more rubber material dampers, such as silicone dampers. Other examples of material for the damping element(s) include nitrile rubber (NBR) and EPDM rubber.

The damping element(s) may provide shock absorption for one or more of the reflector device, the light source, such as the circuit board, the spacer ring, and the spacer plate.

The UV transparent area, such as the covering element, may be arranged on the first damping element, the second damping element, and/or the third damping element. The first damping element, the second damping element, and/or the third damping element may provide damping from the UV-transparent area, such as impacts on the UV-transparent area and/or thermal expansion of the UV-transparent area, such as thermal expansion of the UV-transparent material.

In one or more example decontamination devices, one or more of, such as each, reflector cavity has a depth (extension along center axis) larger than 4 mm such as in the range from 7 mm to 50 mm, e.g. in the range from 9 mm to 25 mm. In one or more example decontamination devices, the reflector cavities have a depth of 11 mm, e.g. allowing suitable control of the light beams from the respective LEDs of the light source.

In one or more example decontamination devices, one or more of, such as each, reflector cavity has a maximum diameter larger than 5 mm, such as in the range from 7 mm to 40 mm, e.g. in the range from 15 mm to 25 mm. In one or more example decontamination devices, the reflector cavities have a maximum diameter of about 20 mm.

In one or more exemplary reflectors, the reflector has a reflector diameter in the range from 4 cm to 10 cm, such as about 55 mm.

In one or more example decontamination devices, respective reflector cavities/reflector openings of reflector device are aligned with respective light emitting elements of the light source, e.g. such that light from a given light emitting element/LED is emitted at and/or through a corresponding reflector opening of a reflector cavity.

The decontamination device comprises an interface. The interface may also be denoted as the first interface. The interface may comprise an activation and/or deactivation arrangement, such as an on/off mechanism, for the light source. The interface may comprise one or more of a button, such as a push button, a switch button, a sliding mechanism, and/or a turning mechanism such as a turning ring. The turning mechanism may for example be spring loaded, such as comprising one or more springs. The turning mechanism may comprise two springs, such that the turning ring may be pressed, turned, and/or rotated in both directions. The interface may provide an operation mechanism for the user to operate the decontamination device.

In one or more example decontamination devices, the decontamination device comprises a spacer plate. The spacer plate may be arranged at a distal surface of the light source. In other words, the light source, such as the circuit board may be arranged between the spacer ring and the spacer plate. In other words, the decontamination device comprises a spacer plate arranged between the circuit board and the first part of the handle part.

In one or more example decontamination devices, the interface comprises an electrical switch. The electrical switch may comprise a reed switch and/or a push button. The spacer plate may comprise a first opening for at least a portion of the electrical switch may contact the light source, such as the circuit board, through the first opening. In one or more example decontamination devices, the decontamination device comprises a spacer ring. The spacer ring may be arranged between the light guide and the handle part. In other words, the spacer ring may be arranged between the light guide and the light source, such as between the UV reflector and the circuit board. The spacer ring may comprise one or more alignment openings, such as a first alignment opening and a second alignment opening in a proximal surface of the spacer ring. In one or more embodiments, the spacer ring comprises a third alignment opening in a proximal surface of the spacer ring. In one or more embodiments, the alignment openings may be through holes in the spacer ring. The spacer ring may provide an adjustment of the height of the circuit board, and in turn of the first set of UV-C LEDs and/or the second set of LEDs. This may be advantageous since the decontamination device may be adapted for different type of LEDs, which provides flexibility for the decontamination device. In other words, the spacer ring may provide adjustment in the optical positioning of the light source with regard e.g. to the light guide.

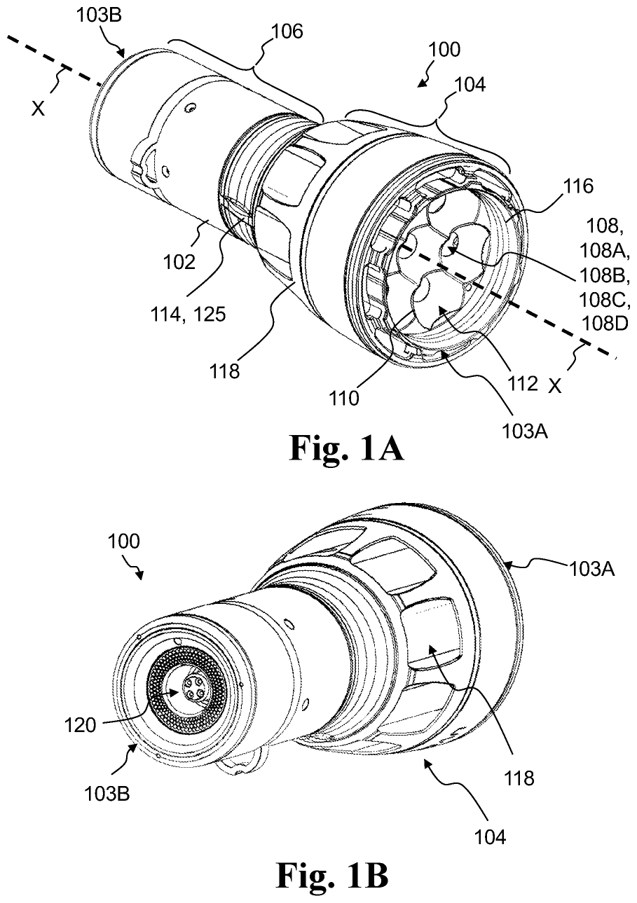

FIG. 1A and FIG. 1B show different perspective views of an exemplary decontamination device. The decontamination device 100 comprises a housing 102 having a first end 103A and a second end 103B and comprising a head part 104 and a handle part 106; a light source 108 configured to emit UV-C light; a light guide 110 for guiding light from the light source towards a UV-transparent area 112 in the head part 104 of the housing; and an interface 114.

The decontamination device 100 optionally comprises a coupling device 116 for coupling the decontamination device to a port of a UV shield or a port of a container of a decontamination system.

The head part 104 comprises a heat sink 118 for cooling the light source 110 during operation.

The decontamination device 100 optionally comprises second interface 120 as illustrated comprising a four-terminal connector at the second end 103B. The second interface 120 is a charging interface and/or a configuration interface for the decontamination device 100.

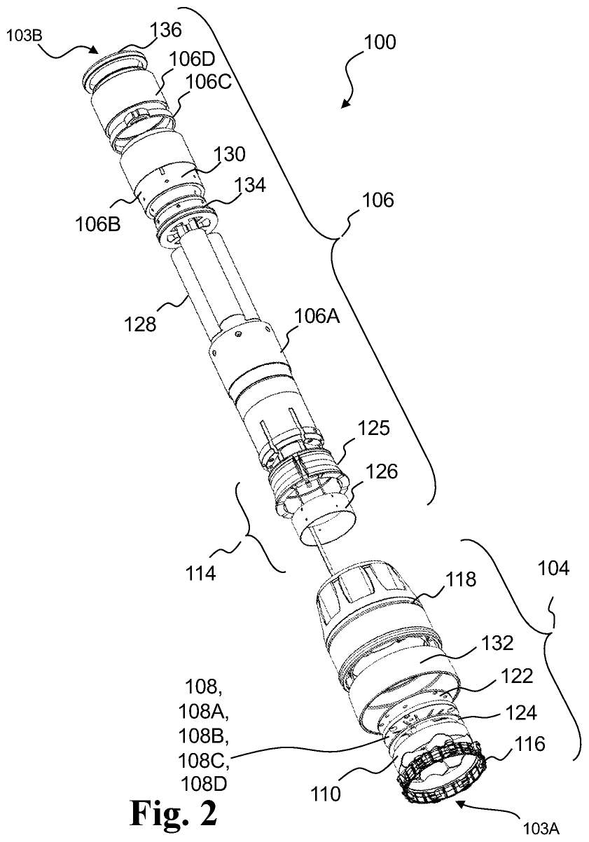

FIG. 2 shows an exploded view of decontamination device 100. The decontamination device 100 optionally comprises a spacer plate 122 distal to or on the distal surface of the light source 108. In other words, the light source 108 may be arranged between the space plate 122 and the first end 103A. The decontamination device 100 optionally comprises a spacer ring 124 proximal to or on the proximal surface of the light source 108. In other words, the spacer ring 124 may be arranged between the light source 108 and the first end 103A. The (first) interface 114 comprises a rotation ring element 125 and an electrical switch 126, the rotation ring element 125 being configured to rotate in relation to the first handle part 106A/handle part 106. The electrical switch 126 detects the rotation of the rotation ring element 125 and provides an input signal to the light source/light controller for controlling the decontamination device/light source based on the first input signal from the electrical switch/interface. The handle part 106 may comprise a first handle part 106A, a second handle part 106B, and optionally a third handle part 106C. The handle part optionally comprise handle coating 106D.

Preferably, the decontamination device comprises a power source or battery pack 128 for feeding power to the electrical components of the decontamination device 100. The power source may be rechargeable e.g. via the second interface 120.

The decontamination device comprises a light controller 130 e.g. mounted or attached to second handle part 106B. The light controller 130 is connected to the light source 108 and the interface and is optionally configured to control the light source based on user input via the interface. The decontamination device 100, e.g. head part 104 may comprise a context ring 132. The decontamination device 100, e.g. handle part 106 may comprise one or more of a connecting element 134 and a protection ring 136.

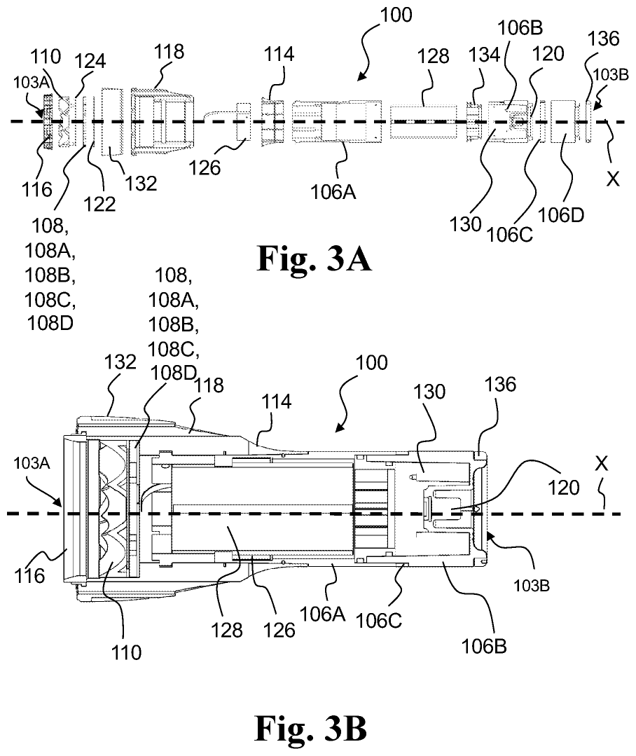

FIG. 3A and FIG. 3B show respective cross-sections for exploded view and assembled view of exemplary decontamination device. When assembled, the light source 108 is arranged inside and in contact with heat sink 118 for transferring heat from the light source to the heat sink 118. The heat sink 118 may form an outer surface of the head part 104. In other words, the heat sink may have an outer surface exposed towards the surroundings, i.e. the heat sink is not covered by, e.g. a protective layer of rubber or polymer. Thus, thereby improved heat exchange between the heat sink 118 and the surroundings may be provided, in turn facilitating a reduced heat sink temperature during use.

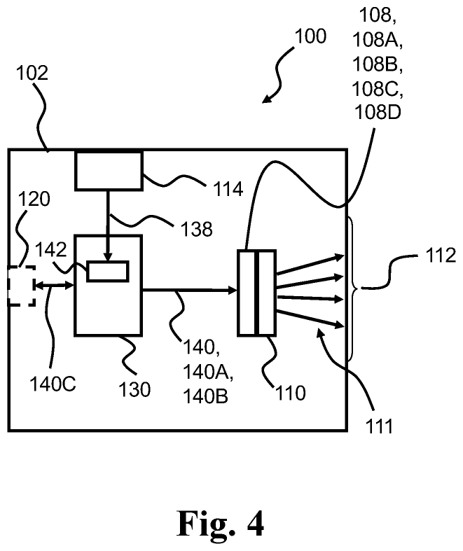

FIG. 4 is a schematic block diagram of an exemplary decontamination device, such as decontamination device 100. The decontamination device 100 comprises a light controller 130. The light controller 130 is connected to the interface 114 for receiving user input via the interface 114. The interface 114 provides an input signal 138 to the light controller 130. The light controller 130 is connected to light source 108, 108A, 108B, 108C, 108D for controlling the light source 108, 108A, 108B, 108C, 108D, such as one or more sets of LEDs of the light source, by control signal 140. The light source 108, 108A, 108B, 108C, 108D emits UV light (illustrated with arrows 111), such as UV-C light, that is guided by light guide 110 towards UV-transparent area 112 based on the control signal 140. The light controller 130 may be separate from the light source 108, 108A, 108B, 108C, 108D or integrated in the light source 108. The light controller 130 may be integrated in the interface 114.

The light controller 130 is configured to apply one or more light schemes, e.g. a first light scheme and/or a second light scheme, to the light source, e.g. in accordance with a user input via the interface 114.

The light controller 130 is optionally configured to apply a first light scheme to the light source, e.g. in response to a first user input via the interface. To apply the first light scheme comprises to activate the first set of UV-C light emitting diodes (LEDs) of the light source and emit UV-C light and/or to activate the second set of light emitting diodes (LEDs) of the light source and emit UV-A light and/or visible light.

The light controller 130 is optionally configured to apply a second light scheme to the light source. The second light scheme may be different from the first light scheme, e.g. in optical power from the first set of UV-C light emitting diodes (LEDs) of the light source and/or duration of the light schemes.

In one or more exemplary decontamination devices, to apply the second light scheme comprises only to activate the first set of UV-C light emitting diodes (LEDs) of the light source. In other words, the second set of LEDs (and/or other LEDs) may be turned off or dimmed in the second light scheme. In one or more exemplary decontamination devices, to apply the second light scheme comprises only to activate the second set of LEDs. In other words, the first set of LEDs (and/or other LEDs) may be turned off or dimmed in the second light scheme. This allows for different applications of the decontamination device to different tasks heavily increasing the user flexibility of the decontamination device.

The light controller 130 may be configured to strobe the first set of UV-C light emitting diodes, e.g. in the first light scheme and/or in the second light scheme. In other words, to activate the first set of UV-C light emitting diodes of the light source, e.g. in the first light scheme and/or in the second light scheme, optionally comprises strobing the first set of UV-C light emitting diodes. In one or more exemplary decontamination devices, strobing the first set of UV-C light emitting diodes comprises operating the first set of UV-C light emitting diodes with a first duty cycle having a first period, e.g. in the range from 1 ms to 100 ms, such as in the range from 5 ms to 30 ms, e.g. about 10 ms, 15 ms, or 20 ms. In other words, the light controller may be configured to strobe the first set of UV-C light emitting diodes with a first duty cycle. The first duty cycle may be larger than 20%, e.g. 50%.

The first light scheme has a duration less than 2 minutes, e.g. less than 1 minute or even in the range from 1 second to 30 seconds, such as in the range from 1 second to 15 seconds.

The light controller 130 may be configured to strobe the second set of light emitting diodes, e.g. in the first light scheme and/or in the second light scheme. In other words, to activate the second set of light emitting diodes of the light source, e.g. in the first light scheme and/or in the second light scheme, optionally comprises strobing the second set of light emitting diodes. In one or more exemplary decontamination devices, strobing the second set of light emitting diodes comprises operating the second set of light emitting diodes with a second duty cycle having a second period, e.g. in the range from 1 ms to 100 ms, such as in the range from 5 ms to 30 ms, e.g. about 10 ms, 15 ms, or 20 ms. The second duty cycle may be larger than 20%, such as 50%.

In one or more exemplary decontamination devices, the first pulse of the first duty cycle is shifted in relation to the second pulse of the second duty cycle, e.g. the first pulse (or first active time) of the first duty cycle may be during the second off time (or second deactive time) of the second duty cycle and/or the second pulse (or second active time) of the second duty cycle may be during the first off time (or first deactive time) of the first duty cycle. This may lead to an improved heat distribution in the decontamination device and/or more evenly distributed load on the power source.

The second light scheme optionally has a duration less than 2 minutes, e.g. less than 1 minute or even in the range from 1 second to 30 seconds, such as in the range from 1 second to 15 seconds.

The light controller 130 may be configured to activate a third light emitting diode of the light source configured to emit visible light, e.g. in the first light scheme and/or in the second light scheme. In other words, to apply the first light scheme and/or the second light scheme optionally comprises to activate a third light emitting diode of the light source configured to emit visible light.

The light controller 130 may be configured to activate, e.g. including strobing with a first duty cycle, the first set of UV-C light emitting diodes for a first primary time period, e.g. in the first light scheme and/or in the second light scheme. In other words, to activate the first set of UV-C light emitting diodes of the light source, e.g. in the first light scheme and/or in the second light scheme, optionally comprises to activate, e.g. including strobing with a first duty cycle, the first set of UV-C light emitting diodes for a first primary time period. The first duty cycle may be constant or vary during the first light scheme and/or in the second light scheme

The light controller 130 may be configured to activate, e.g. including strobing with a second duty cycle, the second set of light emitting diodes for a second primary time period, e.g. in the first light scheme and/or in the second light scheme. In other words, to activate the second set of light emitting diodes of the light source, e.g. in the first light scheme and/or in the second light scheme, optionally comprises to activate, e.g. including strobing with a second duty cycle, the second set of light emitting diodes for a second primary time period. The second duty cycle may be constant or vary during the first light scheme and/or in the second light scheme. The second primary time period may start before or at the same time as the start of the first primary time period. Thus, visible light may be emitted before and/or during UV-C light being emitted in turn notifying the user on an upcoming UV-C light emission. Thus, the safety for the user is increased.

The first light scheme and/or the second light scheme, may comprise a first primary pause and/or a first visible time period before the first primary time period. The light controller 130 may be configured to, e.g. in a first visible time period of the first light scheme and/or the second light scheme, activate, e.g. including strobing optionally with fixed, increasing or decreasing duty cycle, the second set of LEDs (second duty cycle) and/or the third LED (third duty cycle). The light controller may be configured to, in a first visible time period of the first light scheme and/or the second light scheme, activate, e.g. including strobing optionally with fixed, increasing or decreasing duty cycle, a light source of the second interface. The first visible time period may have a duration less than 10 seconds, such as in the range from 1 second to 8 seconds.

The first primary time period may be less than 1 second or in the range from 1 second to 10 seconds, e.g. when the first set of UV-C LEDs are activated multiple times during the first light scheme and/or second light scheme, e.g. in first secondary time period and/or first tertiary time period. The first primary time period may be less than 2 minutes, e.g. less than 1 minute or even in the range from 1 second to 30 seconds. The first primary time period may be in the range from 1 second to 15 seconds.

The second primary time period may be less than 1 second or in the range from 1 second to 10 seconds, e.g. when the second set of LEDs are activated multiple times during the first light scheme and/or second light scheme, e.g. in second secondary time period and/or second tertiary time period. The second primary time period may be less than 2 minutes, e.g. less than 1 minute or even in the range from 1 second to 30 seconds. The second primary time period may be in the range from 1 second to 15 seconds.

The light controller 130 may be configured to, e.g. in the first light scheme and/or in the second light scheme, activate the first set of UV-C light emitting diodes for a first secondary time period and/or a first tertiary time period, e.g. after the second primary time period. In other words, to activate the first set of UV-C light emitting diodes of the light source optionally comprises to activate the first set of UV-C light emitting diodes for a first secondary time period and/or a first tertiary time period, e.g. after the second primary time period. In one or more exemplary decontamination devices, the first secondary time period and/or the first tertiary time period is less than 1 second or in the range from 1 second to 10 seconds. In one or more exemplary decontamination devices, the first secondary time period and/or the first tertiary time period is less than 2 minutes, e.g. less than 1 minute or even in the range from 1 second to 30 seconds. The first secondary time period and/or the first tertiary time period may be in the range from 1 second to 15 seconds.

The light controller 130 may be configured to, e.g. in the first light scheme and/or in the second light scheme, activate the second set of light emitting diodes for a second secondary time period and/or a second tertiary time period, e.g. after the second primary time period. In other words, to activate the second set of light emitting diodes of the light source optionally comprises to activate the second set of light emitting diodes for a second secondary time period and/or a second tertiary period, e.g. after the first secondary time period. In one or more exemplary decontamination devices, the second secondary time period and/or the second tertiary period is less than 1 second or in the range from 1 second to 10 seconds. In one or more exemplary decontamination devices, the second secondary time period and/or the second tertiary period is less than 2 minutes, e.g. less than 1 minute or even in the range from 1 second to 30 seconds. The second secondary time period and/or the second tertiary period may be in the range from 1 second to 15 seconds.

The light controller 130 is optionally configured to activate the first set of UV-C light to emit UV-C light with a power larger than 10 mW, such as in the range from 20 mW to 400 mW. In other words, to activate the first set of UV-C light emitting diodes of the light source optionally comprises to emit UV-C light with a power larger than 10 mW, such as in the range from 20 mW to 400 mW. To activate the first set of UV-C light emitting diodes of the light source optionally comprises to emit UV-C light with a power in the range from 1 mW to 20 mW or in the range from 20 mW to 50 mW. In one or more exemplary decontamination devices, to activate the first set of UV-C light emitting diodes of the light source optionally comprises to emit UV-C light with a power in the range from 50 mW to 1,000 mW or even larger than 1,000 mW. For example, to activate the first set of UV-C light emitting diodes of the light source optionally comprises to emit UV-C light with a power in the range from 100 mW to 900 mW, 200 mW to 800 mW, 300 mW to 700 mW, and/or 350 mW to 600 mW. For example, to activate the first set of UV-C light emitting diodes of the light source optionally comprises to emit UV-C light with a power of 250 mW, 300 mW, 350mW 400 mW, 450 mW, 500 mW, and/or 550 mW.

The light controller 130 is optionally configured to activate a second set of light emitting diodes of the light source to emit UV-A light with a power larger than 200 mW, e.g. in the range from 500 mW to 5 W. In other words, to activate a second set of light emitting diodes of the light source optionally comprises to emit UV-A light with a power larger than 200 mW, e.g. in the range from 500 mW to 5 W. To activate a second set of light emitting diodes of the light source may comprise to emit UV-A light with a power in the range from 1 mW to 200 mW or in the range from 200 mW to 500 mW or larger than 500mW for each second LED. In one or more exemplary decontamination devices, to activate a second set of light emitting diodes of the light source comprises to emit UV-A light with a power in the range from 500 mW to 2 W, such as in the range from 1 W to 1.7 W, e.g. in the range from 1.2 W to 1.4 W, optionally at 700 mA, or even larger than 2 W for each UV-A LED of the second set of UV-A light emitting diodes.

The light controller 130 optionally comprises an input detector 142 configured to detect input from the interface 114. The light controller 130 is configured to control the light source 108, 108A, 1088, 108C, 108D based on the input detected by the input detector. For example, the light controller 130 may be configured to apply the first light scheme in response to a first input via the interface 114 and/or to apply the second light scheme in response to a second input via the interface 114. The control signal 140 or a plurality of control signals 140A, 140B from the light controller controls activation and deactivation of the LEDs of the light source 108, 108A, 1088, 108C, 108D. For example, the control signal 140 may control, e.g. activate and/or de-activate, the first set of UV-C LEDs and the second set of LEDs. In other words, the control signal 140 may be a common control signal for the LEDs of light source 108, 108A, 108B, 108C, 108. In one or more exemplary decontamination devices, a first control signal 140A may control the first set of UV-C LEDs and a second control signal 140B may control, e.g. activate and/or de-activate, the second set of LEDs.

In one or more exemplary decontamination devices, the light controller 130 controls a light source of the second interface 120 via a third control signal 140C.

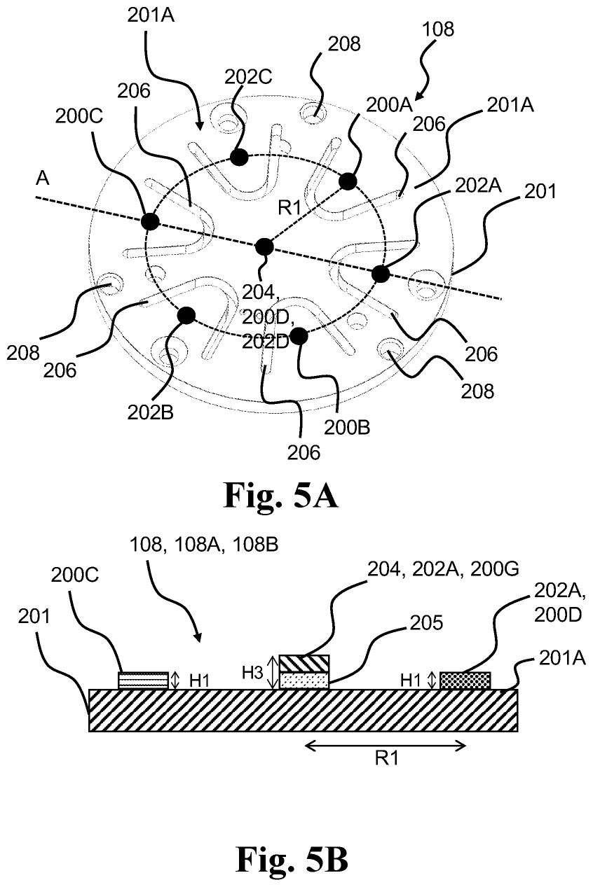

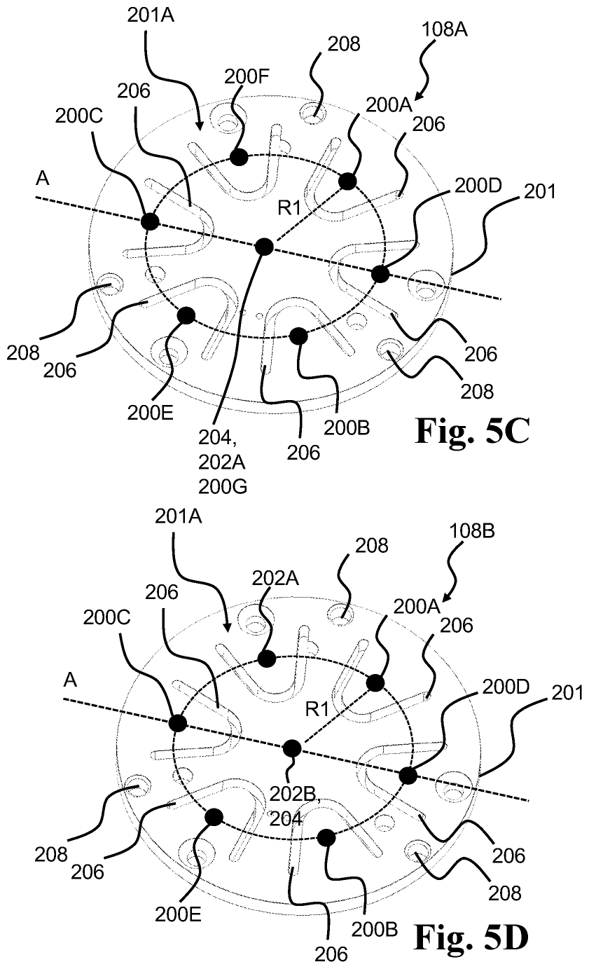

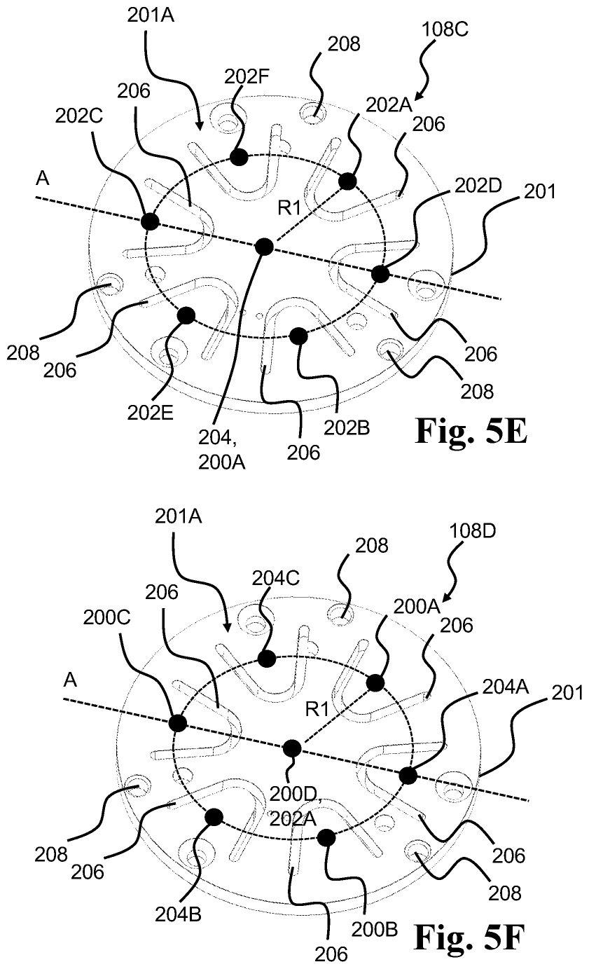

FIGS. 5A-5D show different views of exemplary light sources.

FIG. 5A shows a perspective view of a light source 108 comprising a circuit board 201. The light source 108 comprises a first set of light elements comprising a first primary light element 200A, a first secondary light element 200B, and a first tertiary light element 200C mounted on a proximal surface 201A of the circuit board 201. The light elements 200A, 200B, 200C are UV-C light emitting diodes.

The light source 108 comprises a second set of light elements comprising one or more second light elements. The second set of light elements optionally comprises a second primary light element 202A, a second secondary light element 202B, and a second tertiary light element 202C, mounted on proximal surface 201A of the circuit board 201. The light elements 202A, 202B, 202C may be UV-A light emitting diodes.

The light source 108 comprises a center diode being a first quaternary light element 200D (UV-C LED), a second quaternary light element 202D (UV-A LED), or a third light element 204, such as a third LED, configured to emit visible light, such as one or more of blue light, red light, yellow light, and green light, or a combination thereof. The third light emitting diode 204 may be a blue or red LED. A light source emitting a combination of UV-C light and visible light (third LED 204 or UV-A LEDs 202) is advantageous in that the visible light may indicate to the user, which parts of a surface or item that are exposed to UV-C light, which in turn provides a decontamination device that is safe to use.

The LEDs 200A, 200B, 200C, 202A, 202B, and 202C are evenly distributed around the center diode 200D, 202D, or 204 positioned in a centre part of the circuit board 201 at first radial distance R1 from the center diode. The first radial distance R1 may be in the range from 1cm to 5 cm. Each first LED (200A, 200B, 200C) neighbours two second LEDs (202A, 202B, 202C) and each second LED neighbours two first LEDs (200A, 200B, 200C) thereby providing a more even distribution of first light (UV-C) from first LEDs (200A, 200B, 200C) and second light (UV-A) (202A, 202B, 202C) from second LEDs in the light from the decontamination assembly.

The light source 108 optionally comprises a second set of light emitting diodes mounted on the circuit board 201. The second set of LEDs of light source comprises three LEDs 202A, 202B, 202C, each LED optionally configured to emit UV light comprising UV-A light and/or visible light. The light source 108, such as the second set of light emitting diodes, may be configured to emit visible light e.g. having a wavelength in the range of 380 nm to 740 nm. The light source 108, such as the second set of light emitting diodes, may be configured to emit UV light comprising UV-A light e.g. having a (peak) wavelength in the range of 320 nm to 380 nm. The light source, such as the second set of light emitting diodes, may be configured to emit UV-A light, e.g. light in the range 360 nm to 400 nm. For example, the upper limit of the range of UV-A light, such as 380 nm, may be visible to the human eye.

In one or more example decontamination devices, the second set of light emitting diodes comprises one or a plurality of UV-A light emitting diodes. For example, the second set of light emitting diodes may be UV-A light emitting diodes and optionally comprises one or a plurality of LEDs, such as two, three, four, five, six or more LEDs. The second set of light emitting diodes may comprise a second primary LED 202A, a second secondary LED 202B, and optionally a second tertiary LED 202C. The second set of UV-A light emitting diodes may comprise a second quaternary LED and/or a second quinary LED. The second set of UV-A light emitting diodes may comprise six or more LEDs including a second senary LED. A single second LED may constitute the second set of light emitting diodes, i.e. the second set of LEDs may comprise one LED, such as one UV-A LED.

The circuit board 201 optionally comprises one or more cut-outs 206, such as a first set of cut-outs. The one or more cut-outs 206 may be through openings through the circuit board 201, such that the heat generated from the light elements circumferentially arranged about the center LED may not be conducted passed the cut-out. Thus, heat generated by circumferentially arranged LEDs may be led away from the centre of the circuit board. The one or more cut-outs 206 may be provided around the light elements 200A, 200B, 200C, 200D, 200E, 200F, 202A, 202B, and/or 202C. The one or more cut-outs 206 may substantially insulate thermally each light element, such as each LED from each other. The one or more cut-outs 206 may substantially insulate thermally a light emitting diode, LED, for example a third light element 204, such as a third light emitting diode, from one or more UV LEDs, for example from the first primary light element 200A, the first secondary light element 200B, and/or the first tertiary light element 200C. Further, the one or more cut-outs 206 may substantially direct the heat conduction from the circuit board 201 to the heat sink, e.g. by blocking the heat conduction towards the centre of the circuit board 201. The one or more cut-outs 206 may be substantially V-shaped and/or arc-shaped. The one or more cut-outs 206 may be arranged such that the open part of the V is oriented in direction of the heat sink, e.g. away from the centre of the circuit board 201.

FIG. 5B is a cross sectional view along the axis A shown in FIG. 5A of example light sources 108, 108A (FIG. 5C), 108B (FIG. 5D). Light source 108, 108A, 108B optionally comprises a spacer 205, e.g. arranged between the circuit board 201 and center LED (LED 204 for light source 108, LED 200G, 202A, or 204 for light source 108A, or LED 204 for light source 108B). Thereby a precise positioning of the center LED may be provided, which in turn allows improved control of the height difference between the center LED and circumferentially arranged LEDs, such as the height difference between LED 204 and first/second LEDs 200A, 200B, 200C, 202A, 202B, and 202C. By controlling a height of the center LED, the area exposed by the center LED may be controlled, e.g. to ensure that a first area exposed/illuminated/irradiated with light from the first set of LEDs 200A, 200B, 200C is smaller than a third area exposed/illuminated/irradiated with light from the center LED/third LED 204. The center LED may thereby have a third height H3, i.e. the height of the spacer 205 combined with the height of the center LED. The circumferentially arranged LEDs including first tertiary LED 200C and second primary LED 202A may have a height H1 as shown in FIG. 5B. A first height of first LEDs 200A, 200B, 200C may when mounted on the circuit board 201 be different from a second height of second LEDs 202A, 202B, 202C.

In one or more example decontamination devices, the first set of UV-C LEDs 200A, 200B, 200C are mounted in a first height H1 on the circuit board 201 and the third LED 204 is mounted in a third height H3 on the circuit board 201, wherein the third height H3 is different from the first height H1, such as larger than the first height H1.

In one or more example decontamination devices, the first set of UV-C light emitting diodes may comprise at least three UV-C LEDs, e.g. between three and twenty UV-C light emitting diodes, such as in the range from four to ten UV-C light emitting diodes.

In one or more example decontamination devices, the second set of (optionally UV-A) light emitting diodes may comprise at least three UV-A LEDs, e.g. between three and twenty UV-A light emitting diodes.

The light source 108 optionally comprises one or more alignment openings, such as three alignment openings 208 for accommodating at least partly one or more protrusions of the light guide. Thereby accurate alignment of LEDs and the light guide reflector cavities is provided for. Alternatively or in combination, light source 108 (or light sources 108A, 108B) optionally comprises one or more alignment protrusions protruding radially and/or proximally (from proximal surface of circuit board) from the light source.

FIG. 5C is a perspective view of an exemplary light source 108A. The first set of light emitting elements/diodes comprises a first quaternary LED 200D and/or a first quinary LED 200E. The first set of light emitting elements/diodes comprises optionally comprises six or more LEDs including a first senary LED 200F. The light source 108A may thereby comprise six UV-C LEDs, such as the first primary LED 200A, the first secondary LED 200B, the first tertiary LED 200C, the first quaternary LED 200D, the first quinary LED 200E, and the first senary LED 200F.

The first LEDs 200A, 200B, 200C, 200D, 200E, and 200F are evenly distributed around a center LED, such as third LED 204 (visible light, such as blue light), second primary LED 202A (UV-A), or first septenary LED 200G (UV-C), positioned in a centre part of the circuit board 201, e.g. at first radial distance R1 from the center LED 204. The first radial distance R1 may be in the range from 10 mm to 50 mm.

FIG. 5D is a perspective view of an exemplary light source 108B. As shown in FIG. 5D, the first set of UV-C light emitting diodes may comprise a first quaternary LED 200D and/or a first quinary LED 200E. The first set of UV-C light emitting diodes may comprise five or more LEDs. The light source may thereby comprise five UV-C LEDs, such as the first primary LED 200A, the first secondary LED 200B, the first tertiary LED 200C, the first quaternary LED 200D, and the first quinary LED 200E, one UV-A LED, such as the second primary LED 202A, and one visible light LED, such as the third LED 204.

In other words, the first set of UV-C light emitting diodes may be distributed, e.g. evenly distributed, around the third LED. Thus, the third LED may be a center LED. The second set of UV-A light emitting diodes may be distributed, e.g. evenly distributed around the third light emitting diode. First LEDs and second LEDs may be alternately arranged around the third light emitting diode. In other words, second LEDs may be respectively arranged between two first LEDs and/or first LEDs may be respectively arranged between second LEDs.

In one or more example decontamination devices, the first set of UV-C light emitting diodes are mounted on the circuit board 201 with a first radial distance R1 from a centre of the circuit board, e.g. from the third light emitting diode 204. The first radial distance R1 may be in the range from 10 mm to 40 mm, such as in the range from 15 mm to 30 mm.

FIG. 5E shows an exemplary light guide 108C with second LEDs 202A, 202B, 202C, 202D, 202E, and 202F evenly distributed around a center LED, such as third LED 204 (visible light, such as blue light), or first primary LED 200A (UV-C), positioned in a centre part of the circuit.

FIG. 5F shows an exemplary light guide 108D with first LEDs 200A, 200B, 200C and third LEDs 204A, 204B, 204C, evenly distributed around a center LED, such as first quaternary LED 200D (UV-C) or second primary LED 202A, positioned in a centre part of the circuit.

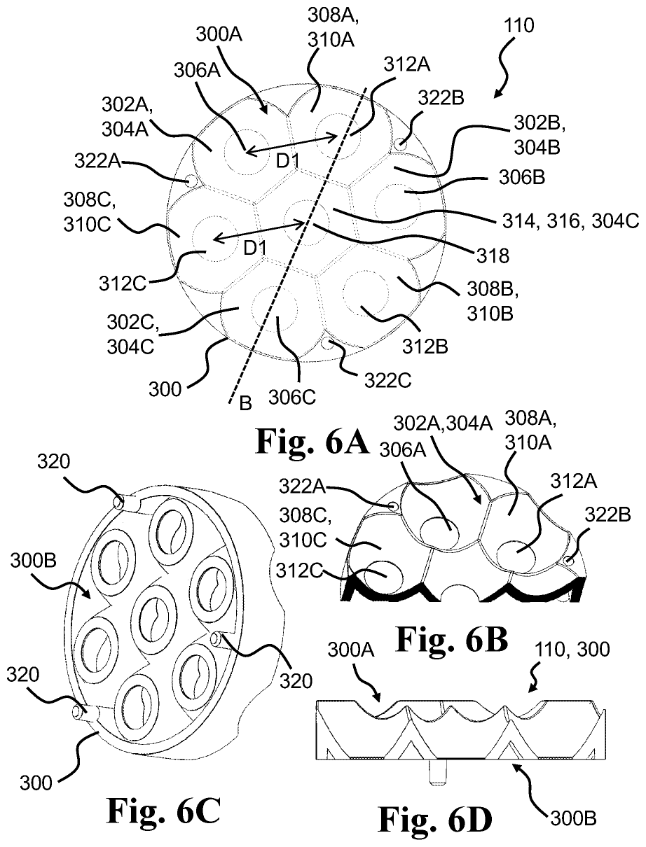

FIGS. 6A-6D show different views of an exemplary light guide. FIG. 6A is a proximal view of the light guide 110. FIG. 6B is a perspective cross-sectional view of the light guide 110 seen from a proximal side. FIG. 6C is a perspective view of the light guide 110 seen from the distal side. FIG. 6D is a cross-sectional view of the light guide 110 along the axis B shown in FIG. 6A.

The light guide 110 comprises a reflector device 300, such as a UV reflector device, having a reflector structure in a proximal surface 300A of the reflector device 300. The reflector structure may comprise one or more, such as a plurality of reflector cavities. The reflector cavities may comprise a first set of reflector cavities and optionally a second set of reflector cavities. The reflector cavities may comprise a third reflector cavity or a third set of reflector cavities. The reflector cavity 300 may be used with any of light guides 108, 108A, 108B, 108C,108D, 108E merely changing the naming of the cavities to correspond to the naming of the LEDs, e.g. first quaternary cavity corresponds to first quaternary light element, etc.

The first set of reflector cavities comprises one or more first reflector cavities including a first primary reflector cavity 302A having a first primary reflector surface 304A, and optionally a first secondary reflector cavity 302B having a first secondary reflector surface 304B. The first set of reflector cavities may include a first tertiary reflector cavity 302C having a first tertiary reflector surface 304C, and optionally a first quaternary reflector cavity 302D having a first quaternary reflector surface 304D. First reflector cavities may be aligned with respective first light emitting diodes of the light source. Accordingly, the number of reflector cavities in the first set of reflector cavities may be the same as the number of LEDs in the first set of LEDs.

The second set of reflector cavities comprises one or more second reflector cavities including a second primary reflector cavity 308A having a second primary reflector surface 310A, and optionally a second secondary reflector cavity 308B having a second secondary reflector surface 310B. The second set of reflector cavities may include a second tertiary reflector cavity 308C having a second tertiary reflector surface 310C, and optionally a second quaternary reflector cavity 308D having a second quaternary reflector surface 310D. Second reflector cavities may be aligned with respective second light emitting diodes of the light source. Accordingly, the number of reflector cavities in the second set of reflector cavities may be the same as the number of LEDs in the second set of LEDs.

A reflector cavity has a reflector surface. The reflector surfaces of the reflector cavities or at least part thereof may be metallic surfaces, such as aluminium, gold, copper, silver, platinum, or a metal alloy comprising one or more of aluminium, gold, copper, silver, and platinum. Aluminium or an alloy comprising aluminium may be preferred as reflector surface material.

In one or more exemplary decontamination devices, first reflector surfaces, such as the first primary reflector surface 304A and/or the first secondary reflector surface 304B are metallic surfaces. The first tertiary reflector surface 304C may be a metallic surface.

In one or more exemplary decontamination devices, second reflector surface(s), such as the second primary reflector surface 310A and/or the second secondary reflector surface 310B are metallic surfaces. The second tertiary reflector surface 310C may be a metallic surface.

The light guide/UV reflector device 300 may be configured to reflect and/or direct the light emitted from the light source towards a UV-transparent area in the head part of the housing for irradiating a surface to be decontaminated by the decontamination device.

The UV reflector device 300 may comprise a reflector cavity for each LED in the light source. In other words, the number of reflector cavities may correspond to the number of light emitting diodes or elements of the light source.

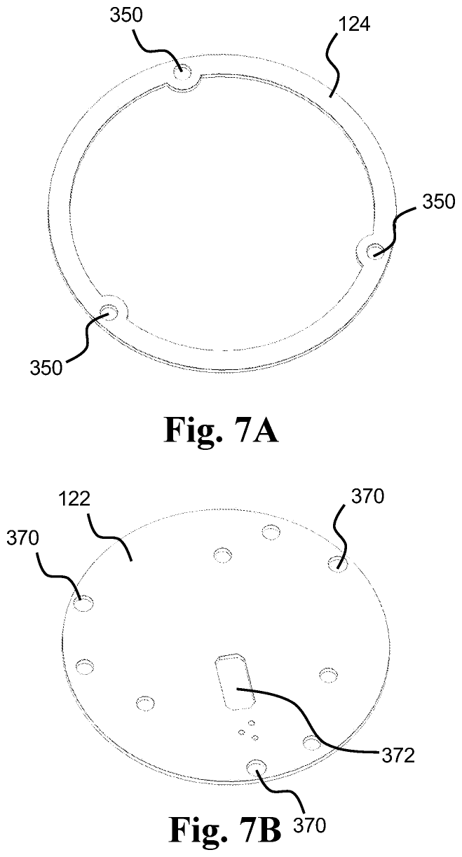

FIG. 7A and 7B show an exemplary spacer ring and an exemplary spacer plate of a decontamination device. In one or more example decontamination devices, the decontamination device comprises a spacer plate 122. The spacer plate 122 may be arranged at a distal surface of the light source. In other words, the light source, such as the circuit board may be arranged between the spacer ring 124 and the spacer plate 122. In other words, the decontamination device comprises a spacer plate 122 arranged between the circuit board and the first part of the handle part.

The spacer plate 122 may comprise a first opening 372 for at least a portion of the electrical switch may contact the light source, such as the circuit board, through the first opening 372. The spacer ring 124 may comprise one or more alignment openings 350, such as a first alignment opening and a second alignment opening in a proximal surface of the spacer ring. In one or more embodiments, the spacer ring comprises a third recess in a proximal surface of the spacer ring. In one or more embodiments, the recesses may be through the spacer ring. The spacer ring may provide an adjustment of the height of the circuit board, and in turn of the first set of UV-C LEDs and/or the second set of LEDs. This may be advantageous since the decontamination device may be adapted for different type of LEDs, which provides flexibility for the decontamination device. In other words, the spacer ring and/or the spacer plate may provide adjustment in the optical positioning of the light source with regard e.g. to the light guide.

In one or more example decontamination devices, the light guide, such as the reflector device 300, may comprise one or more protrusions and/or recesses in a distal surface of the reflector device, as can be seen on FIGS. 6C-D. For example, the reflector device 300 optionally comprises one or more protrusions, such as two protrusions, three protrusions, and/or four protrusions extending from the distal surface 300B of the reflector device 300.

The protrusion(s) may be alignment protrusions 320 for aligning the light guide 110 and the light source. In an assembled state of the decontamination device, the one or more alignment protrusions 320 in the distal surface 300B of the reflector device 300 may be at least partly accommodated in one or more alignment openings of the light source and optionally alignment openings 370 in the spacer plate 122 and/or alignment openings 350 in the spacer ring 124. In other words, the reflector device 300, the circuit board 201, the spacer plate 122, and/or the spacer ring 124 may form an assembly that is substantially connected to each other, when the one or more alignment protrusions 320 of the reflector device 300 are accommodated in the one or more alignment openings 208, 350, 370. Thereby, the LEDs of the light source and the reflector cavities may be aligned to precisely arrange the LEDs in relation to the reflector cavities, thereby improving intensity of the UV-light emitted through the UV-transparent area.

In one or more example decontamination devices, the reflector surfaces, such as the first reflector surfaces of the first set of reflector cavities and/or the second reflector surface(s) of the second set of reflector cavities, are concave surfaces, such as concave aspheric surfaces. In other words, a cross section of a reflector cavity may be concave aspheric in shape, as can be seen in FIG. 6D. The reflector structure may therefore act as an aspherical concave optic, such as lens, in the decontamination device. For example, the plurality of reflector cavities may be overlapping, such as overlapping cones, to provide the aspherical concave shape of the reflector structure. The reflector device may be configured to reflect at least 50% of the light emitted from the light source, at least 60% of the light emitted from the light source, at least 70% of the light emitted from the light source, at least 80% of the light emitted from the light source, at least 90% of the light emitted from the light source, and/or at least 98% of the light emitted from the light source.

The reflector device 300 may comprise respective openings, such as a first set of openings and/or a second set of openings, in the respective reflector surfaces, such as first openings in respective first reflector cavities and/or second openings in respective second reflector cavities, e.g. for forming light passages between the reflector cavities and a distal side of the reflector device and/or to allow positioning respective LEDs in or at respective reflector cavities for provision of light to be reflected by the reflector surfaces.

In one or more example decontamination devices, the reflector device 300 comprises a first primary reflector opening 306A in the first primary reflector surface 304A, e.g. for forming a first primary light passage between the first primary reflector cavity 302A and a distal side of the reflector device 300 and/or to allow positioning a first primary LED in or at the first primary reflector cavity 302A for provision of light to be reflected by the first primary reflector surface 304A. The first primary reflector opening 306A may be circular, e.g. with a diameter in the range from 2 mm to 15 mm. The reflector device may comprise a first secondary reflector opening 306B in the first secondary reflector surface 304B, e.g. for forming a first secondary light passage between the first secondary reflector cavity 302B and a distal side of the reflector device 300 and/or to allow positioning a first secondary LED in or at the first secondary reflector cavity 302B for provision of light to be reflected by the first secondary reflector surface 304B. The first secondary opening may be circular, e.g. with a diameter in the range from 2 mm to 15 mm.

The reflector device 300 may comprise a second primary reflector opening 312A in the second primary reflector surface 310A, e.g. for forming a second primary light passage between the second primary reflector cavity 308A and a distal side of the reflector device 300 and/or to allow positioning a second primary LED in or at the second primary reflector cavity 308A for provision of light to be reflected by the second primary reflector surface 310A. The second primary reflector opening 312A may be circular, e.g. with a diameter in the range from 2 mm to 15 mm.

In one or more example decontamination devices, the reflector structure comprises four, five, six, seven or more reflector cavities. In a reflector structure with at least four reflector cavities, at least three, such as four, five, six, or more reflector cavities including the first primary reflector cavity 302A, the first secondary reflector cavity 302B, and the second primary reflector cavity 308A, may be distributed around a third reflector cavity 314 also denoted a center reflector cavity.

In one or more example decontamination devices, the first set of reflector cavities and optionally the second set of reflector cavities are, e.g. evenly, distributed around a third reflector cavity 314.

The reflector structure 300 may comprise one or more of a first tertiary reflector cavity 302C of the first set of reflector cavities, a second secondary reflector cavity 308B and a second tertiary reflector cavity 308C of the second set of reflector cavities, and/or a third reflector cavity 314.

The first tertiary reflector cavity 302C has a first tertiary reflector surface 304C, the second secondary reflector cavity 308B has a second secondary reflector surface 310B, the second tertiary reflector cavity 308C has a second tertiary reflector surface 310C, and the third reflector cavity 314 optionally has a third reflector surface 316.

The reflector device 300 may comprise a first tertiary reflector opening 306C in the first tertiary reflector surface 304C, wherein the first tertiary opening 306C is optionally circular with a diameter in the range from 2 mm to 15 mm. The reflector device 300 may comprise a second secondary reflector opening 312B in the second secondary reflector surface 310B, wherein the second secondary opening 312B is optionally circular with a diameter in the range from 2 mm to 15 mm. The reflector device 300 may comprise a second tertiary reflector opening 312C in the second tertiary reflector surface 310C, wherein the second tertiary reflector opening 312C (sixth opening) is optionally circular with a diameter in the range from 2 mm to 15 mm. The reflector device 300 may comprise a third reflector opening 318 in the third reflector surface 316, wherein the third reflector opening 318 is optionally circular with a diameter in the range from 2 mm to 15 mm.

In one or more example decontamination devices, a center-to-center distance D1 between two neighbouring reflector cavities is in the range from 10 mm to 25 mm.

In one or more example decontamination devices, the light source is configured to emit visible light and/or UV light comprising one or both of UV-C light and UV-A light.

In one or more example decontamination devices, the reflector device 300 comprises a plurality of damping recesses including a first (damping) recess 322A and a second (damping) recess 322B in the proximal surface 300A of the reflector device 300, such as of the reflector body. In one or more example decontamination devices, the decontamination device comprises a first damping element (not shown) at least partly accommodated in the first recess 322A, and a second damping element (not shown) at least partly accommodated in the second recess 322B. The reflector device 300 optionally comprises a third (damping) recess 322C in the proximal surface 300A of the reflector body. In one or more example decontamination devices, the decontamination device comprises a third damping element (not shown) at least partly accommodated in the third recess 322C.

In one or more example decontamination devices, one or more of, such as each, reflector cavity has a depth in the range from 10 mm to 50 mm.

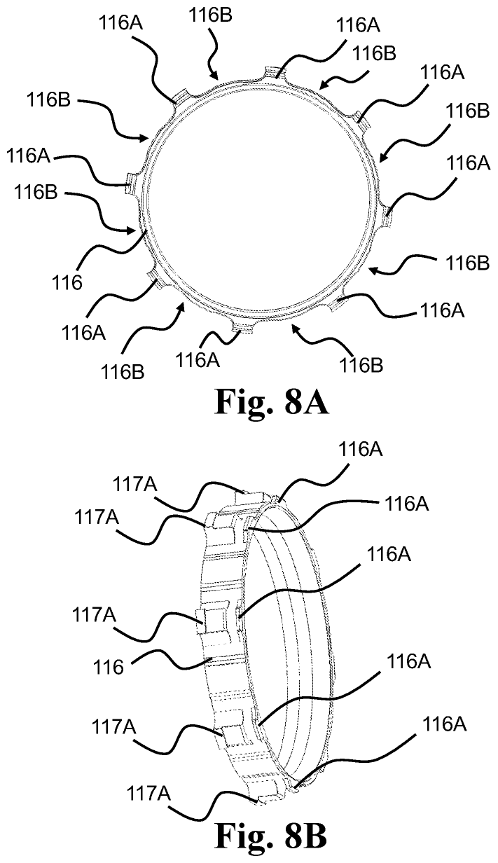

FIGS. 8A and 8B shows different views of an exemplary coupling device. The coupling device is embodied as a coupling ring 116. The coupling device/coupling ring 116 comprises a plurality of rods or protrusions 116A extending radially outwards and being evenly distributed along an outer periphery 117 of the coupling device/coupling ring 116. The plurality of protrusions 116A may comprise at least three protrusions, such as three, four, five, six, seven, eight or more protrusions.

The coupling device/coupling ring 116 comprises a plurality of recesses 116B in a radially outward facing surface of the coupling device/coupling ring 116. The recesses 116B are evenly distributed along an outer periphery of the coupling device/coupling ring 116 and comprises at least three recesses, such as three, four, five, six, seven, eight or more recesses.

The coupling ring optionally comprises coupling elements 117A configured for attaching the coupling ring 116 to heat sink 118 of head part. The coupling elements 117A may be circumferentially aligned with protrusions 116A. A threading on coupling ring 116 may be provided for attaching the coupling ring 116 to heat sink 118 of head part. Thereby, the coupling ring may be configured for locked attachment to the heat sink of the head part, e.g. by means of coupling elements 117A forming locking protrusions. The protrusions 116A and recesses 116B form a bayonet coupling part for coupling to a corresponding bayonet coupling part of a UV shield or a container, i.e. for coupling to a corresponding bayonet coupling part of a port.

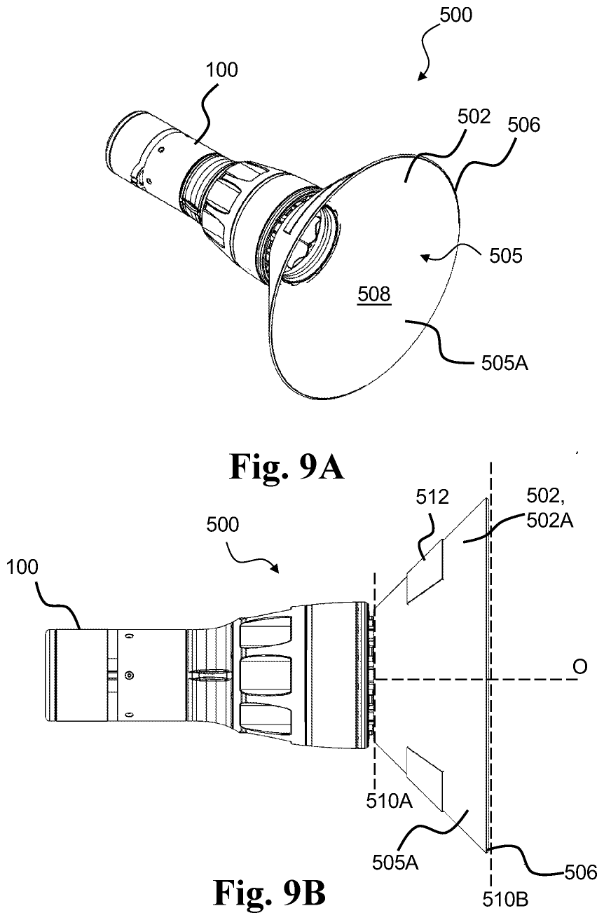

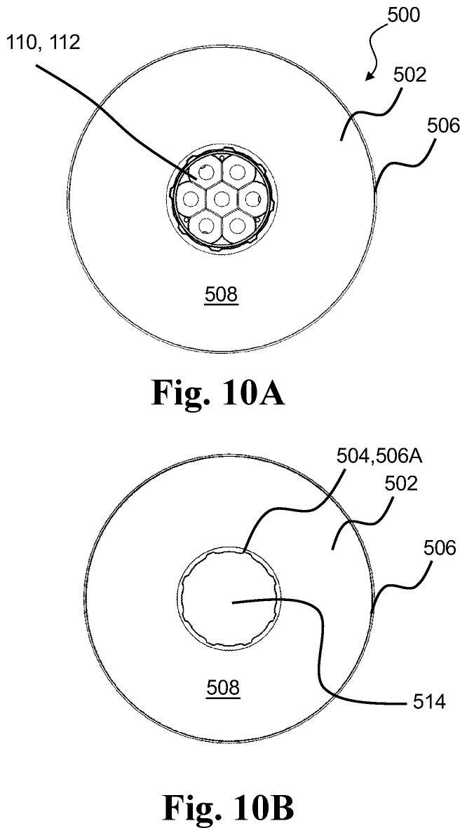

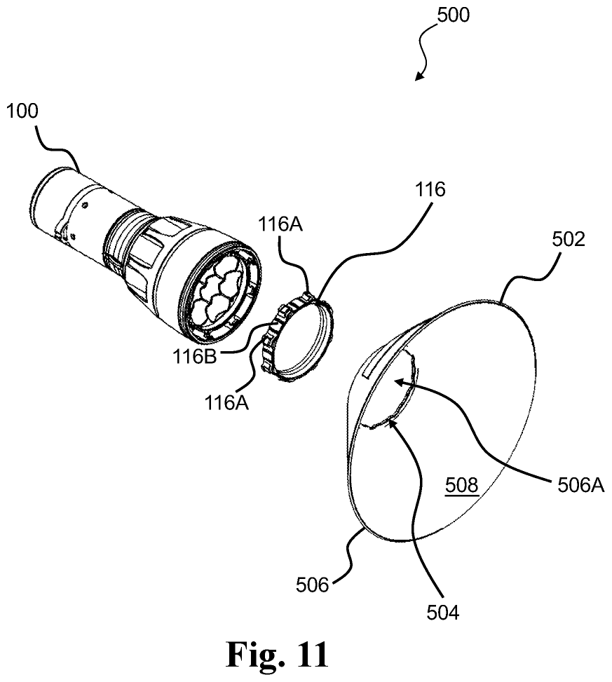

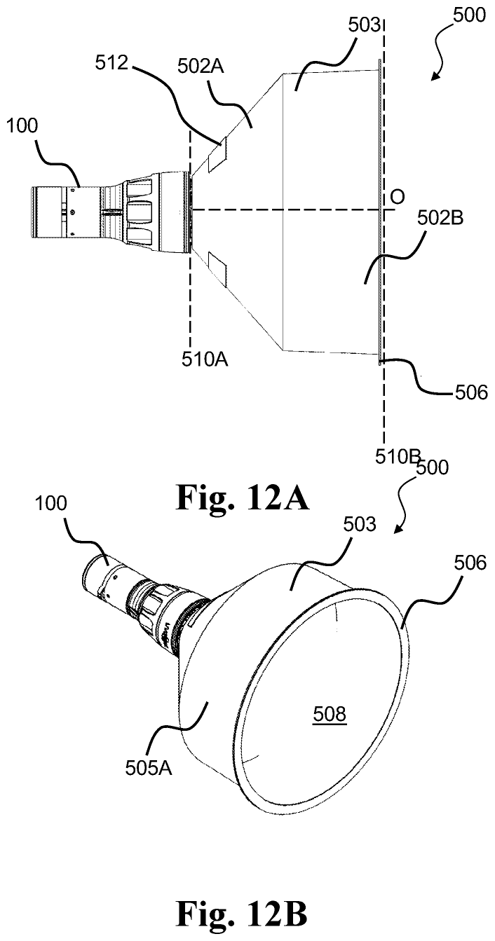

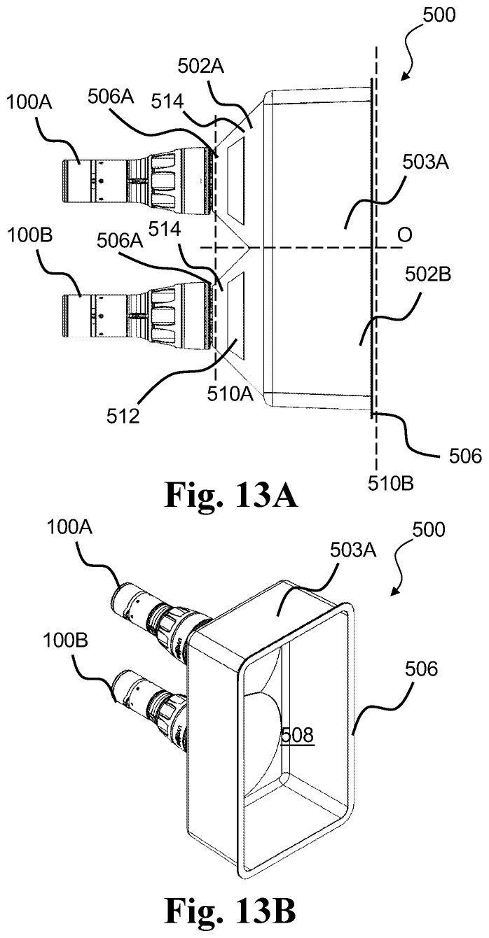

FIGS. 9A-13B show different views of an exemplary decontamination assembly, such as decontamination assembly 500. FIG. 9A is a perspective view of an exemplary decontamination assembly 500 comprising a UV shield 502. FIG. 9B is a side-view of an exemplary decontamination assembly 500 comprising a UV shield 502. FIG. 10A is a proximal view of an exemplary decontamination assembly 500. FIG. 10B is a proximal view of an exemplary UV shield 502. FIG. 11 is an exploded perspective view of an exemplary decontamination assembly. FIG. 12A is a side-view of an exemplary decontamination assembly 500 comprising a UV shield 503. FIG. 12B is a perspective view of an exemplary decontamination assembly 500 comprising UV shield 503. FIG. 13A is a side-view of an exemplary decontamination assembly 500 comprising a UV shield 503A with two ports. FIG. 13B is a perspective view of an exemplary decontamination assembly 500 comprising UV shield 503A.

FIGS. 9A-11 shows a decontamination assembly 500 comprising decontamination device and a UV shield 502. The decontamination device may be decontamination device 100 and/or a decontamination device as disclosed herein. The decontamination device optionally comprises a coupling device 116 for coupling, e.g. releasably coupling, the decontamination device/head part to a port 506A arranged in or at first end 510A of the UV shield 502. The coupling device 116 may be arranged in the head part, e.g. at the first end of the housing. In other words, the head part may comprise the coupling device. In one or more exemplary decontamination devices, the coupling device 116 is a separate coupling element, e.g. attached to the heat sink 118. The coupling device 116 may be integrated in the heat sink.

In one or more example decontamination assemblies and/or decontamination devices, the UV shield 502 is made of a UV absorbing polymer material. For example, the UV shield 502 may be made of polycarbonate, polypropylene, an acrylic, polyethylene, ABS, POM, PEEK, or DelRin. The UV shield may be made of metal or coated with metal or metal alloy on inner and/or outer surface.

In one or more example decontamination assemblies and/or decontamination devices, the UV shield 502, 503, 503A has an inner volume 505 and comprises one or more walls 505A defining the inner volume 505. The UV shield 502 comprises a rim 506 defining an opening 508 for accessing the inner volume. The opening 508 of the UV shield 502 allows for arranging an item within the inner volume 505, e.g. by the rim 506 contacting a surface on which the item is placed. Thereby, the UV shield 502 shields a user from UV light from the light source during decontamination of the item.

In one or more example decontamination assemblies and/or decontamination devices, the UV shield 502 may comprise one or more reinforcement parts 512. The reinforcement part may provide the UV shield 502 with an improved structural rigidity, e.g. when coupling or attaching the decontamination device 100 to the UV shield. The reinforcement part may avoid that the UV shield 502 bends or changes in shape, when the decontamination device 100 is attached and/or during use of the decontamination assembly.

In one or more example decontamination assemblies and/or decontamination devices, the opening 508 has an area of in the range from 25 cm2 to 500 cm2. The opening 508 may have a largest extension in the range from 5 cm to 50 cm. The opening 508 may have a smallest extension in the range from 1 cm to 30 cm.

In one or more example decontamination assemblies and/or decontamination devices, the rim is circular or polygonal, e.g. as in FIGS. 9A-12B. In other words, the opening may be circular, oval, polygonal or other shape which facilitates arranging an item within the inner volume of the UV shield. The opening may be rectangular optionally with rounded corners as can be seen in FIGS. 13A-13B.

The UV shield 502, 503, 503A has an inner volume for at least partly accommodating an item to be decontaminated. The inner volume may have a volume in the range from 50 cm³ to 5,000 cm³.

The UV shield 502, 503, 503A comprises one or more walls defining the inner volume. The UV shield may comprise a first part 502A and optionally a second part 502B, e.g. as in FIGS. 12A-13B. The first part 502A may be conical. The second part 502B may be cylindrical. The UV shield 502 may extend from a first end 510A to a second end 510B along a center axis O. The port 506A of the UV shield 502 may be arranged at the first end 510A. The opening 508/rim 506 of the UV shield 502 may be arranged at the second end 510B of the UV shield. The first part 502A may extend from the first end 510A towards the second end 510B. The second part 502B may extend from the second end 510B towards the first end 510A. A first inner surface of the first part 502A may partly define the inner volume and optionally has an increasing cross-sectional area towards the second end 510B, e.g. along the center axis O of the UV shield. The first part/first inner surface 502A may be conical. A second inner surface of the second part 502B may partly define the inner volume and optionally has a constant, decreasing, or increasing cross-sectional area towards the second end, e.g. along the center axis O. The second part/second inner surface 502B may be cylindrical, e.g. have a constant and circular, oval, polygonal cross-sectional area, e.g. along the center axis O.

In one or more example decontamination assemblies and/or decontamination devices, the UV shield 502 is conical or at least partly conical.

The UV shield has a port 506A, also denoted first port, for releasably coupling the decontamination device 100 and the UV shield 502, 503, 503A.

FIG. 12A and 12B show a UV shield 503 having a conical first part 502A and a cylindrical second part 502B. The first part 502A of UV shield 503 has a length in the range from 2 cm to 15 cm, such as about 6 cm as shown. The second part 502B of UV shield 503 has a length in the range from 2 cm to 15 cm, such as about 6 cm as shown.

The UV-shield 502 may comprise a plurality of ports including a first port and a second port, e.g. as in FIGS. 13A-13B. Thereby, multiple decontamination devices may be coupled to the UV-shield and/or a decontamination device can be used in different coupling arrangements depending on the item to be decontaminated which in turn increases the user flexibility.

A port may have an opening for passage of UV light from the decontamination device into the inner volume. For example, the (first) port of the UV shield may have a (first) opening with an area in the range from 10 cm² to 50 cm². The (first) opening of the (first) port may be circular or substantially circular. The port may comprise a coupling structure, such as a bayonet coupling device, for releasably coupling the port/UV shield to the decontamination device.

FIG. 13A and 13B show an exemplary decontamination assembly comprising UV shield 503A. The UV shield 503A comprises second part 502B with substantially rectangular cylindrical shape with a length in the range from 2 cm to 15 cm, such as about 12 cm as shown. The UV-shield 503A has two ports 506A for coupling multiple decontamination devices or at least for coupling a decontamination device in different positions. The two ports are arranged at first ends of respective first and second cone structures 514 of the UV shield (first part).

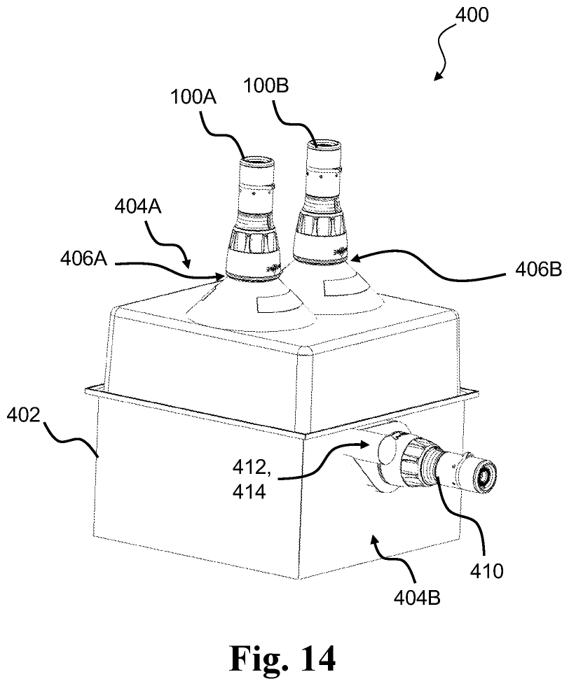

FIG. 14 is a perspective view of an exemplary decontamination system, such as the decontamination system 400.

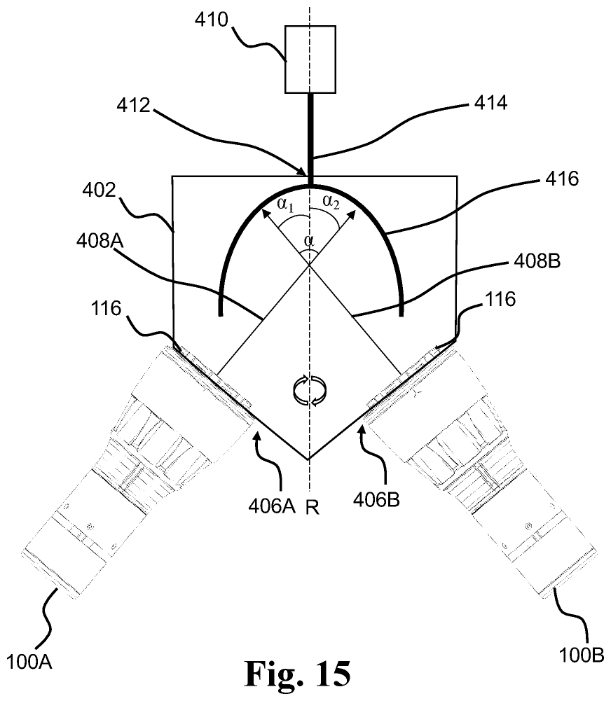

FIG. 15 is a schematic view of an exemplary decontamination system.

The decontamination system 400 comprises a container 402 having an inner volume defined by at least one wall including a first wall 404A, and a carrier device 416 rotatably arranged about a rotation axis R in the inner volume. The decontamination system 400 optionally comprises a motor 410 configured to rotate the carrier device 416. The decontamination system 400, e.g. the container 402, may comprise a first port 406A in the first wall 404A of the container 402, wherein the first port 406A is configured to couple a decontamination device, such as a first decontamination device 100A, to the container 402. The decontamination system 400, e.g. the container 402, may comprise a second port 406B in a wall of the container 402, such as the first wall 404A wherein the second port 406B is configured to couple a decontamination device, such as a second decontamination device 1008, to the container 402. It is to be understood that a description of a port of a UV-shield 502 is also applicable to a port of the decontamination system 400 and vice versa. A UV-shield as described herein, such as UV shield 503A, may form a part of the decontamination system 400 e.g. as part of a two-part container 402.

The coupling device 116 of the decontamination device 100A, 1008 may be configured for releasably coupling the decontamination device 100A, 1008 to a port of the container 402. The coupling device 116 of the decontamination device 100A, 1008 may comprise a coupling element. The coupling element may be ring-shaped optionally encircling the UV-transparent area. In other words, the coupling element may have an opening forming the UV-transparent area. The coupling element may be a coupling ring. The coupling ring may be attached to the heat sink.

In one or more exemplary decontamination systems, the first port 406A and/or the second port 406B comprises a bayonet coupling device for releasably coupling a decontamination device 100A, 100B to the container 402.

In one or more exemplary decontamination systems, the first port 406A comprises a first closure element for closing a first opening of the first port 406A. The second port 406B may comprise a second closure element for closing a second opening of the second port 406B.

In one or more exemplary decontamination systems, the container 402 is made of a UV absorbing polymer material. For example, the container 402 may be made of the material of the container 402 and/or the UV shield may be transparent or at least partly transparent for visible light. Thereby, a user can follow the decontamination procedure during decontamination or the item/surface.

In one or more exemplary decontamination systems, the first port 406A is configured to attach a decontamination device, e.g. the first decontamination device 100A, such that a first beam from the decontamination device, e.g. the first decontamination device 100A, coupled to the first port 406A is in a first direction 408A (parallel to central axis X of the decontamination device 100A). The second port 406B may be configured to attach a decontamination device, such as a second decontamination device 100B, such that a second beam from the second decontamination device 100B coupled to the second port 406B is in a second direction 408B (parallel to central axis X of the decontamination device 100B). The first direction 408A and the second direction 408B may form a beam angle α larger than 30°, such as in the range from 45° to 180°. The beam angle α may be in the range from 60° to 120°. Thereby an improved exposure of the item to be decontaminated may be provided in the decontamination system 400.

In one or more exemplary decontamination systems, a first angle α₁ between the rotation axis R and the first direction is less than 60°, such as less than 30°. The first angle α₁ between the rotation axis R and the first direction may be in the range from 15° to 60°. The first angle α₁ may, e.g. in a single-port container, be in the range from 60° to 90°, such as in the range from 75° to 90°, e.g. about 90°.

In one or more exemplary decontamination systems, a second angle α₂ between the rotation axis R and the second direction is larger than 30°. The second angle α₂ between the rotation axis R and the second direction may be in the range from 15° to 90°, such as in the range from 30° to 60°.

In one or more exemplary decontamination systems, the container 402 comprises a rotation opening 412, and wherein the carrier device comprises a shaft 414 rotatably arranged in the rotation opening 412. The shaft 414 may be a telescopic arm. A rotation opening 412 with a rotatably arranged shaft 414 therein, allows rotation of the carrier device by a motor 410 arranged outside the container 402.

In one or more exemplary decontamination systems, the carrier device 416 comprises a frame part and a seat part, wherein the seat part comprises a seat configured for accommodating an item to be decontaminated, wherein the seat part is made of a seat material made of a UV transparent material, such as silicone. The carrier device 416 may be connected to the shaft 414. Different type of carrier devices may be mounted in the container of the decontamination system, e.g. different type of carrier devices may be mounted to the shaft of the decontamination system. In some embodiments, the shaft may act as a carrier device.

The item may be press-fitted in the seat, and UV transparent material may allow for a 360° exposure of the item during rotation in turn allowing a more efficient decontamination procedure, e.g. by eliminating the need for a double-position decontamination procedure where the item is first decontaminated in a first position on the carrier device 416 and then rearranged and then secondly decontaminated in a second position on the carrier device 416.

In one or more exemplary decontamination systems, the decontamination system 400 comprises a first decontamination device 100A and optionally a second decontamination 100B, wherein the first decontamination device 100A is configured to releasably couple to the first port 406A and/or the second port 406B, and optionally the second decontamination device 100B is configured to releasably couple to the second port 406B. The first decontamination device 100A and/or the second decontamination device 100B may be a decontamination device 100 as disclosed herein.

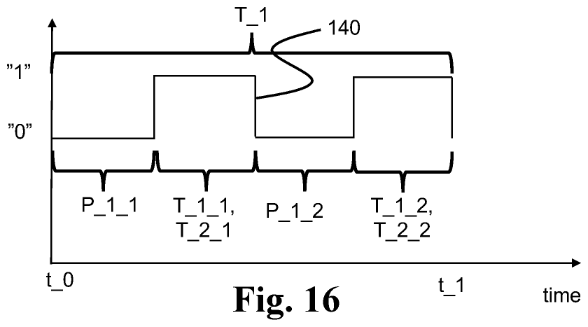

FIG. 16 shows a control signal 140 of an exemplary first light scheme applied to the light source of the decontamination device. The control signal 140 controls both the first set and the second set of LEDs of the light source. In FIG. 16, the control signal 140 activates or switches on the first set of LEDs and the second set of LEDs when the control signal 140 goes high or logical “1” and deactivates or turns off the first set of LEDs and the second set of LEDs when the control signal 140 goes low or logical “0”.

The first light scheme starts at time t_0 optionally in response to a user providing a first input via interface 114 and ends at time t_1 and having a duration T_1 in the range from 1 second to 30 seconds. The first light scheme optionally starts with and comprises a first primary pause P_1_1, e.g. ranging from 1 second to 5 seconds. During P_1_1, a third control signal 140C optionally activates a light source of second interface e.g. with fixed or increasing duty cycle during P_1_1.

The first light scheme comprises a first primary time period T_1_1 and a second primary time period T_2_1 where the first set of LEDs and the second set of LEDs are activated by control signal, respectively. During T_1_1 and _T_2_1, the first set of LEDs and the second set of LEDs are strobed with respective first duty cycle D_1 and second duty cycle D_2 of 50% and a time period of 10 ms corresponding to 100 Hz. Other duty cycles and/or time periods may be used. The strobing functionality may be included in the control signal 140 by strobing the control signal 140 or embedded via a strobe generator in the light source controlled by the control signal. T_1_1 and T_2_1 may be in the range from 1 to 30 seconds. The first light scheme may end after T_1_1 and T_2_1.

The first light scheme optionally comprises a first secondary pause P_1_2, e.g. ranging from 1 second to 5 seconds, e.g. in order to allow cooling of the LEDs.

The first light scheme optionally comprises a first secondary time period T_1_2 and a second secondary time period T_2_2 where the first set of LEDs and the second set of LEDs are activated by control signal 140, respectively. During T_1_2 and _T_2_2, the first set of LEDs and the second set of LEDs are strobed with respective first duty cycle D_1 and second duty cycle D_2 of 50% and a time period of 10 ms corresponding to 100 Hz. Other duty cycles and/or time periods may be used. T_1_2 and T_2_2 may be in the range from 1 to 30 seconds.

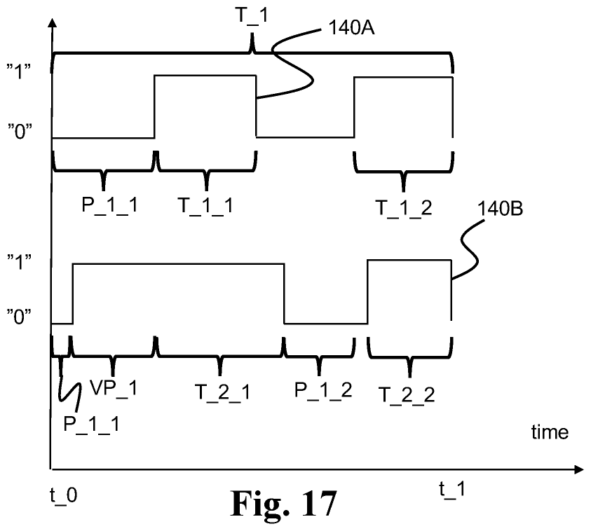

FIG. 17 shows control signals 140A, 14B of an exemplary first light scheme applied to the light source of the decontamination device. The first control signal 140A controls the first set of LEDs of the light source and the second control signal 140B controls the second set of LEDs of the light source. The first control signal 140A activates or switches on the first set of LEDs, when the first control signal 140A goes high or logical “1” and deactivates or turns off the first set of LEDs when the first control signal 140A goes low or logical “0”. The second control signal 140B activates or switches on the second set of LEDs, when the second control signal 140B goes high or logical “1” and deactivates or turns off the second set of LEDs when the second control signal 140B goes low or logical “0”.

The first light scheme starts at time t_0 optionally in response to a user providing a first input via interface 114 and ends at time t_1 and having a duration T_1 in the range from 1 second to 30 seconds. The first light scheme optionally starts with and comprises a first visible time period VP_1 before the first primary time period T_1_1, the first visible time period VP_1 optionally e.g. ranging from 1 second to 5 seconds. During VP_1, a third control signal 140C optionally activates a light source of second interface e.g. with fixed or increasing duty cycle during VP_1. During VP_1, the second control signal 140B activates, e.g. strobes the second set of LEDs, e.g. at reduced power, reduced second duty cycle and/or increased second period.

The first light scheme in FIG. 17 comprises a first primary time period T_1_1 where the first set of LEDs is activated by first control signal 140A, and a second primary time period T_2_1 where second set of LEDs is activated by second control signal 140B. During T_1_1, the first set of LEDs is strobed with a first duty cycle D_1 of between 40% and 60% and a time period of 10 ms corresponding to 100 Hz. During T_2_1, the second set of LEDs is strobed with a second duty cycle D_2 of between 40% and 60% and a time period of 10 ms corresponding to 100 Hz. D_1 and D_2 may the same or different. Separate control signals for each set of LEDs allows increased design flexibility and customization of the light scheme(s) to different decontamination requirements. The strobing functionality may be included in control signals 140A, 140B by strobing the control signal 140A, 140B or embedded via a strobe generator in the light source controlled by the control signal 140A, 140B. T_1_1 and T_2_1 may be in the range from 1 to 30 seconds. The first light scheme may end after T_1_1 and T_2_1.

The first light scheme optionally comprises a first secondary pause P_1_2, e.g. ranging from 1 second to 5 seconds, e.g. in order to allow cooling of the LEDs.

The first light scheme optionally comprises a first secondary time period T_1_2 and a second secondary time period T_2_2 where the first set of LEDs and the second set of LEDs are activated by control signal 140, respectively. During T_1_2 and _T_2_2, the first set of LEDs and the second set of LEDs are strobed with respective first duty cycle D_1 and second duty cycle D_2 of 50% and a time period of 10 ms corresponding to 100 Hz. Other duty cycles and/or time periods may be used. T_1_2 and T_2_2 may be in the range from 1 to 30 seconds.

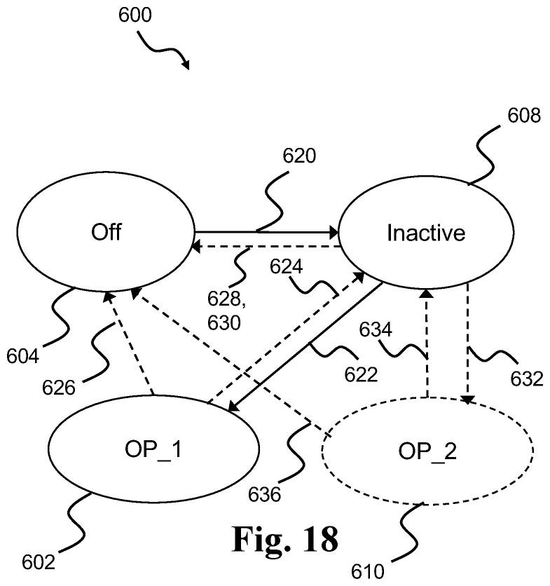

FIG. 18 illustrates an exemplary state diagram 600 of the light controller illustrating operation of the decontamination device.

The decontamination device/light controller is configured to operate in one or a plurality of states including a first operating state 602 and optionally one or more of an off state 604, a lock state (not shown here but later indicated with ref 606), an inactive state 608, and a second operating state 610.

The decontamination device is in the off state 604 when not in use. In the off state 604, the light controller/input detector is configured to detect an on input 620, e.g. consisting of a single on input event, e.g. a first rotate-and-release or a first rotate-hold-and-release input. In response to detection of the on input 620, the light controller moves the decontamination device to the inactive state 608. In the inactive state 608, the light controller optionally activates a light source in the second interface, e.g. by strobing light and or light with an inactive color, and/or emits visible light via one or more LEDs of the light source.

In the inactive state 608, the light controller/input detector is configured to detect a first input 622, such as a first input described herein, e.g. selected from Table 1 above. In response to a detection of the first input 622, the light controller moves the decontamination device to the first operating state 602. In the first operating state, the light controller is optionally configured to apply a first light scheme, such as first light scheme described herein or in relation to FIG. 16 or 17, and/or activate a light source of second interface. Optionally, the light controller is configured to move to the inactive state 608 or the off state 604 from the first operating state 602 after applying the first light scheme or in response to a detection of a stop input as indicated by arrows 624 and 626, respectively. In one or more exemplary decontamination devices, the light controller is configured to move to the inactive state 608 after applying the first light scheme as indicated by arrow 624 and to move to the off state 604 in response to a detection of a stop input as indicated by arrow 626. In one or more exemplary decontamination devices, the light controller is configured to move to the off state 604 after applying the first light scheme as indicated by arrow 626 and to move to the inactive state 608 in response to a detection of a stop input as indicated by arrow 624.

To move to the inactive state 608, e.g. from one or more of the off state 604, the first operating state 602, and the second operating state, optionally comprises to reset a timer.

In the inactive state 608, the light controller/input detector is configured to detect an off input 628 or that the timer reaches a time threshold 630, e.g. of 1 minute. In response to a detection of the off input 628 or the timer reaching a time threshold 630, the light controller optionally moves the decontamination device to the off state 604.

In the inactive state 608, the light controller/input detector is optionally configured to detect a second input 632, such as a second input described herein, e.g. selected from Table 2 above. In response to a detection of the second input 632, the light controller optionally moves the decontamination device to the second operating state 610. In the second operating state, the light controller is optionally configured to apply a second light scheme, such as second light scheme described herein, and/or activate a light source of second interface. Optionally, the light controller is configured to move to the inactive state 608 or the off state 604 from the second operating state 610 after applying the second light scheme or in response to a detection of a stop input as indicated by arrows 634 and 636, respectively. In one or more exemplary decontamination devices, the light controller is configured to move to the inactive state 608 after applying the second light scheme as indicated by arrow 634 and to move to the off state 604 in response to a detection of a stop input as indicated by arrow 636. In one or more exemplary decontamination devices, the light controller is configured to move to the off state 604 after applying the second light scheme as indicated by arrow 636 and to move to the inactive state 608 in response to a detection of a stop input as indicated by arrow 634.

In one or more exemplary decontamination devices, the light controller/input detector may not detect first input and/or second input in a pause period, e.g. of in the range from 1 to 30 seconds after moving to the inactive state 608. Thereby is ensured that the LEDs are not overheated or have sufficient time to cool off before being activated again, in turn leading to longer life time of the decontamination device. In other words, the input detector may be deactivated in a pause period after moving to the inactive state 608 or first input and/or second input may be ignored by the light controller during or in the pause period.

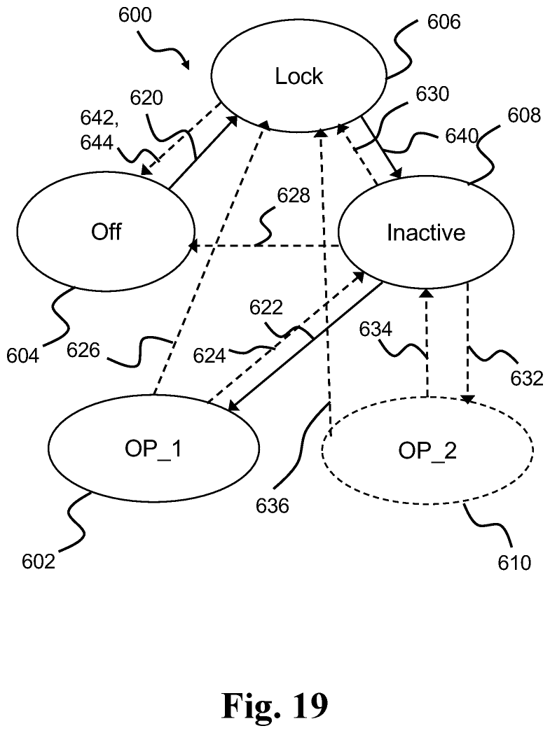

FIG. 19 illustrates an exemplary state diagram 600A of the light controller illustrating operation of the decontamination device.

The decontamination device/light controller is configured to operate in one or a plurality of states including a first operating state 602 and optionally one or more of an off state 604, a lock state 606, an inactive state 608, and a second operating state 610.

The decontamination device is in the off state 604 when not in use. In the off state 604, the light controller/input detector is configured to detect an on input 620, e.g. consisting of a single on input event, e.g. a first rotate-and-release or a first rotate-hold-and-release input. In response to detection of the on input 620, the light controller moves the decontamination device to the lock state 606. In the lock state 606, the light controller optionally activates a light source in the second interface, e.g. by strobing light and/or light with a lock color.

In the lock state 606, the light controller/input detector is configured to detect an unlock input 640, e.g. consisting of a single on input event, e.g. a second rotate-and-release or a second rotate-hold-and-release input. In response to detection of the unlock input 640, the light controller moves the decontamination device to the inactive state 608. In the inactive state 608, the light controller optionally activates a light source in the second interface, e.g. by strobing light and/or light with an inactive color, and/or emits visible light via one or more LEDs of the light source.

In the inactive state 608, the light controller/input detector is configured to detect a first input 622, such as a first input described herein, e.g. selected from Table 1 above. In response to a detection of the first input 622, the light controller moves the decontamination device to the first operating state 602. In the first operating state, the light controller is optionally configured to apply a first light scheme, such as first light scheme described herein or in relation to FIG. 16 or 17 and/or activate a light source of second interface. Optionally, the light controller is configured to move to the inactive state 608 or the lock state 606 from the first operating state 602 after applying the first light scheme or in response to a detection of a stop input as indicated by arrows 624 and 626, respectively. In one or more exemplary decontamination devices, the light controller is configured to move to the inactive state 608 after applying the first light scheme as indicated by arrow 624 and to move to the lock state 606 in response to a detection of a stop input as indicated by arrow 626. In one or more exemplary decontamination devices, the light controller is configured to move to the lock state 606 after applying the first light scheme as indicated by arrow 626 and to move to the inactive state 608 in response to a detection of a stop input as indicated by arrow 624.

To move to the inactive state 608, e.g. from one or more of the off state 604, the first operating state 602, and the second operating state, optionally comprises to reset a timer.

In the inactive state 608, the light controller/input detector is optionally configured to detect an off input 628 and/or that the timer reaches a time threshold 630, e.g. of 1 minute. In response to a detection of the off input 628, the light controller optionally moves the decontamination device to the off state 604 from the inactive state 608. In response to the timer reaching a time threshold 630 in the inactive state, the light controller optionally moves the decontamination device to the lock state 606 from the inactive state or to the off state 604 from the inactive state (not shown).

In the inactive state 608, the light controller/input detector is optionally configured to detect a second input 632, such as a second input described herein, e.g. selected from Table 2 above. In response to a detection of the second input 632, the light controller optionally moves the decontamination device to the second operating state 610. In the second operating state 610, the light controller is optionally configured to apply a second light scheme, such as second light scheme described herein, and/or activate a light source of second interface. Optionally, the light controller is configured to move to the inactive state 608 or the lock state 606 from the second operating state 610 after applying the second light scheme or in response to a detection of a stop input as indicated by arrows 634 and 636, respectively. In one or more exemplary decontamination devices, the light controller is configured to move to the inactive state 608 after applying the second light scheme as indicated by arrow 634 and to move to the off state 604 in response to a detection of a stop input as indicated by arrow 636. In one or more exemplary decontamination devices, the light controller is configured to move to the off state 604 after applying the second light scheme as indicated by arrow 636 and to move to the inactive state 608 in response to a detection of a stop input as indicated by arrow 634.

In one or more exemplary decontamination devices, the light controller/input detector may not detect first input and/or second input in a pause period, e.g. of in the range from 1 to 30 seconds after moving to the inactive state 608. Thereby is ensured that the UV-LEDs are not overheated or have sufficient time to cool off before being activated again, in turn leading to longer life time of the decontamination device. In other words, the input detector may be deactivated in a pause period after moving to the inactive state 608 or first input and/or second input may be ignored by the light controller during or in the pause period.

The present disclosure includes decontamination systems, decontamination devices, and decontamination assemblies according to any of the following items.

Item DD1. A decontamination device comprising:
a housing comprising a head part and a handle part;
a light source configured to emit UV-C light;
a light guide for guiding light from the light source towards a UV-transparent area in the head part of the housing; and
an interface.

Item DD2. Decontamination device according to Item DD1, wherein the light guide comprises a reflector device having a reflector structure, e.g. in a proximal surface of the reflector device, the reflector structure comprising a plurality of reflector cavities comprising a first set of reflector cavities and a second set of reflector cavities, each reflector cavity having a reflector surface, the first set of reflector cavities including a first primary reflector cavity having a first primary reflector surface, and a first secondary reflector cavity having a first secondary reflector surface, and the second set of reflector cavities including a second primary reflector cavity having a second primary reflector surface.

Item DD3. Decontamination device according to Item DD2, wherein the first primary reflector surface is a metallic surface.

Item DD4. Decontamination device according to any of Items DD2-DD3, wherein the reflector device comprises a reflector body and a first layer coated onto the reflector body, wherein the reflector body is made of a body material and the first layer is made of a first reflector material, and wherein the first reflector material comprises a metal being one or more of aluminium, gold, copper, silver, and platinum.

Item DD5. Decontamination device according to any of Items DD2-DD4, wherein the reflector surfaces are concave aspheric surfaces.

Item DD6. Decontamination device according to any of Items DD2-DD5, wherein the reflector device comprises a first primary opening in the first primary reflector surface, wherein the first primary opening is circular with a diameter in the range from 2 mm to 15 mm.

Item DD7. Decontamination device according to any of Items DD2-DD6, wherein the reflector structure comprises seven reflector cavities, wherein six reflector cavities including the first primary reflector cavity, the first secondary reflector cavity, and the second primary reflector cavity, are distributed around a third reflector cavity.

Item DD8. Decontamination device according to any of Items DD2-DD7, wherein a center-to-center distance between two neighbouring reflector cavities is in the range from 10 mm to 25 mm.

Item DD9. Decontamination device according to any of Items DD1-DD8, wherein the light source is configured to emit visible light and/or UV light comprising UV-A light.

Item DD10. Decontamination device according to any of Items DD2-DD9, wherein the reflector device comprises a first recess and a second recess in the proximal surface of the reflector body, wherein the decontamination device comprises a first damping element at least partly accommodated in the first recess, and a second damping element at least partly accommodated in the second recess.

Item DD11. Decontamination device according to any of Items DD2-DD10, wherein each reflector cavity has a depth in the range from 7 mm to 50 mm.

Item DD12. Decontamination device according to any of Items DD1-DD11, wherein the light source comprises: a circuit board; a first set of UV-C light emitting diodes mounted on the circuit board, the first set of UV-C light emitting diodes comprising at least three UV-C light emitting diodes; and optionally a second set of light emitting diodes mounted on the circuit board, wherein the second set of light emitting diodes is configured to emit visible light and/or UV light comprising UV-A light.

Item DD13. Decontamination device according to Item DD12, wherein the first set of UV-C light emitting diodes comprises between three and twenty UV-C light emitting diodes.

Item DD14. Decontamination device according to any of Items DD12-DD13, wherein the second set of light emitting diodes comprises a plurality of UV-A light emitting diodes.

Item DD15. Decontamination device according to any of Items DD12-DD14, wherein the light source comprises a third light emitting diode configured to emit visible light.

Item DD16. Decontamination device according to Item DD15, wherein the UV-C light emitting diodes and the UV-A light emitting diodes are distributed around the third light emitting diode positioned in a center part of the circuit board.

Item DD17. Decontamination device according to any of Items DD12-DD16, wherein the light source has in the range from five to ten light emitting diodes mounted on the circuit board.

Item DD18. Decontamination device according to any of Items DD12-DD17, wherein a center-to-center distance between two neighbouring light emitting diodes is in the range from 10 mm to 25 mm.

Item DD19. Decontamination device according to any of Items DD12-DD18, as dependent on Item DD15, wherein the first set of UV-C light emitting diodes are mounted in a first height on the circuit board and the third light emitting diode is mounted in a third height on the circuit board, wherein the third height is different from the first height.

Item DD20. Decontamination device according to any of Items DD15-DD19, wherein the light source comprises a spacer arranged between the circuit board and the third light emitting diode.

Item DD21. Decontamination device according to any of Items DD12-DD20, wherein the first set of UV-C light emitting diodes are mounted on the circuit board with a first radial distance from a center of the circuit board.

Item DD22. Decontamination device according to any of Items DD1-DD21, the decontamination device comprising a heat sink for cooling the light source, the light source comprising:
a circuit board made of material comprising a first layer of first material with a first thermal conductivity and a first heat capacity; and
a first set of UV-C light emitting diodes mounted on the circuit board, wherein the heat sink is in thermal contact with the first layer and made of a second material with a second thermal conductivity and a second heat capacity, wherein the second heat capacity is larger than the first heat capacity.

Item DD23. Decontamination device according to Item DD22, wherein the first thermal conductivity is larger than the second thermal conductivity.

Item DD24. Decontamination device according to any of Items DD22-DD23, wherein the first material comprises copper.

Item DD25. Decontamination device according to any of Items DD22-DD24, wherein the second material is aluminium.

Item DD26. Decontamination device according to any of Items DD22-DD25, wherein the heat sink forms an outer surface of the head part.

Item DD27. Decontamination device according to any of Items DD22-DD26, wherein the heat sink comprises a coating on the outer surface, the coating having a thickness less than 300 μm.

Item DD28. Decontamination device according to any of Items DD22-DD27, wherein the heat sink comprises a plurality of channels extending parallel to a longitudinal axis of the decontamination device.

Item DD29. Decontamination device according to any of Items DD1-DD28, wherein the decontamination device comprises a light controller connected to the light source for controlling the light source, wherein the interface is connected to the light controller.

Item DD30. Decontamination device according to Item DD29, wherein the light controller is configured to apply a first light scheme to the light source, wherein to apply the first light scheme comprises:
to activate a first set of UV-C light emitting diodes of the light source; and
to activate a second set of light emitting diodes of the light source, wherein the second set of light emitting diodes is configured to emit visible light and/or UV light comprising UV-A light.

Item DD31. Decontamination device according to Item DD30, wherein the light controller is configured to apply a second light scheme to the light source, and wherein the second light scheme is different from the first light scheme.

Item DD32. Decontamination device according to any of Items DD30-DD31, wherein to activate the first set of UV-C light emitting diodes of the light source comprises strobing the first set of UV-C light emitting diodes.

Item DD33. Decontamination device according to any of Items DD30-DD32, wherein the first light scheme has a duration in the range from 1 second to 30 seconds.

Item DD34. Decontamination device according to any of Items DD30-DD33, wherein to activate the second set of light emitting diodes of the light source comprises strobing the second set of light emitting diodes.

Item DD35. Decontamination device according to any of Items DD30-DD34, wherein to apply the first light scheme comprises to activate a third light emitting diode of the light source configured to emit visible light.

Item DD36. Decontamination device according to any of Items DD30-DD35, wherein to activate the first set of UV-C light emitting diodes of the light source comprises to activate the first set of UV-C light emitting diodes for a first primary time period; and wherein to activate the second set of light emitting diodes of the light source comprises to activate the second set of light emitting diodes for a second primary time period.

Item DD37. Decontamination device according to Item DD36, wherein the first primary time period is in the range from 1 second to 10 seconds.

Item DD38. Decontamination device according to any of Items DD36-DD37, wherein the second primary time period is in the range from 1 second to 10 seconds.

Item DD39. Decontamination device according to any of Items DD36-DD38, wherein the first primary time period is separate from the second primary time period.

Item DD40. Decontamination device according to any of Items DD36-DD39, wherein to activate the first set of UV-C light emitting diodes of the light source comprises to activate the first set of UV-C light emitting diodes for a first secondary time period after the second primary time period.

Item DD41. Decontamination device according to any of Items DD36-DD40, wherein to activate the second set of light emitting diodes of the light source comprises to activate the second set of light emitting diodes for a second secondary time period after the first secondary time period.

Item DD42. Decontamination device according to any of Items DD30-DD41, wherein to activate the first set of UV-C light emitting diodes of the light source comprises to emit UV-C light with an optical power in the range from 20 mW to 800 mW

Item DD43. Decontamination device according to any of Items DD30-DD42, wherein to activate a second set of light emitting diodes of the light source comprises to emit UV-A light with an optical power in the range from 500 mW to 2 W.

Item DD44. Decontamination device according to any of Items DD1-DD43, wherein the decontamination device is configured to operate in a plurality of states including a first operating state and one or more of a lock state, an inactive state, and a second operating state.

Item DD45. Decontamination device according to Item DD44 as dependent on Item 29, the light controller comprising an input detector configured to detect a first input comprising a plurality of first input events and, wherein the light controller is configured to, in response to a detection of the first input, move the decontamination device to the first operating state.

Item DD46. Decontamination device according to Item DD45, wherein the light controller is configured to move the decontamination device to the first operating state from the lock state and/or the inactive state.

Item DD47. Decontamination device according to any of Items DD45-DD46, wherein the first input comprises a first primary input event, wherein the first primary input event comprises one or more of a press-and-hold input, a press input, a multi-press input, a rotate-and-hold input, a rotate input, a multi-rotate input, a slide-and-hold input, a slide input, and a multi-slide input.

Item DD48. Decontamination device according to any of Items DD45-DD47, wherein the first input comprises a first secondary input event, wherein the first secondary input event comprises one or more of a press-and-hold input, a press input, a multi-press input, a rotate-and-hold input, a rotate input, a multi-rotate input, a slide-and-hold input, a slide input, and a multi-slide input.

Item DD49. Decontamination device according to any of Items DD45-DD48, wherein the input detector is configured to detect an unlock input, and wherein the light controller is configured to, in response to detection of the unlock input, to move the decontamination device to the inactive state.

Item DD50. Decontamination device according to any of Items DD45-DD49, wherein the input detector is configured to detect a lock input, and wherein the light controller is configured to, in response to detection of the lock input, move the decontamination device to the lock state.

Item DD51. Decontamination device according to any of Items DD45-DD50, wherein the light controller is configured to, in the first operating state, apply a first light scheme and after applying the first light scheme move to the inactive state or the lock state.

Item DD52. Decontamination device according to any of Items DD45-DD51, wherein the input detector is configured to detect a second input and wherein the light controller is configured to, in response to a detection of the second input:
move the decontamination device to a second operating state; and
apply, in the second operating state, a second light scheme.

Item DD53. Decontamination device according to Item DD52, wherein the input detector is configured to, after applying the second light scheme, move the decontamination device to the inactive state or the lock state.

Item DD54. Decontamination device according to any of Items DD45-DD53, wherein the light controller is configured to reset a timer when moving the decontamination device to the inactive state and move the decontamination device to the lock state from the inactive state if the timer reaches a time threshold.

Item DD55. Decontamination device according to any of Items DD45-DD54, wherein the input detector is configured to detect a stop input and wherein the light controller is configured to, in response to detection of the stop input, deactivate the light source and move the decontamination device to the inactive state, the lock state, or the off state.

Item DD56. Decontamination device according to any of Items DD1-DD55, wherein the head part comprises a coupling device for releasably coupling the head part to a port of a UV shield and/or a first port of a container.

Item DD57. Decontamination assembly according to Item DD56, wherein the coupling device comprises a plurality of rods extending radially and/or a plurality of recesses.

Item DD58. Decontamination assembly according to any of Items DD56-DD57, wherein the coupling device comprises a bayonet coupling part for coupling to a corresponding bayonet coupling part of the UV shield and/or the first port of a container.

Item DA1. A decontamination assembly comprising a decontamination device and a UV shield, the decontamination device comprising:
a housing comprising a head part and a handle part;
a light source configured to emit UV-C light;
a light guide for guiding light from the light source towards a UV-transparent area in the head part of the housing; and
an interface,
   wherein the head part comprises a coupling device for releasably coupling the head part to a port of the UV shield.

Item DA2. Decontamination assembly according to Item DA1, wherein the coupling device comprises a plurality of rods extending radially and/or a plurality of recesses.

Item DA 3. Decontamination assembly according to any of Items DA1-DA2, wherein the coupling device comprises a bayonet coupling part for coupling to a corresponding bayonet coupling part of the UV shield.

Item DA 4. Decontamination assembly according to any of Items DA1-DA3, wherein the UV shield is made of a UV absorbing polymer material.

Item DA 5. Decontamination assembly according to any of Items DA1-DA4, wherein the UV shield has an inner volume and comprises a rim defining an opening for accessing the inner volume.

Item DA 6. Decontamination assembly according to Item DA5, wherein the opening has an area of in the range from 25 cm² to 500 cm².

Item DA 7. Decontamination assembly according to any of Items DA5-DA6, wherein the rim is circular or polygonal.

Item DA 8. Decontamination assembly according to any of Items DA5-DA7, wherein the inner volume has a volume in the range from 50 cm³ to 5,000 cm³.

Item DA9. Decontamination assembly according to any of Items DA1-DA8, wherein the UV shield is conical.

Item DA10. Decontamination assembly according to any of Items DA1-DA9, wherein the port of the UV shield has a port opening with an area in the range from 10 cm² to 50 cm².

Item DA11. Decontamination assembly according to any of Items DA1-DA9, wherein the decontamination device is a decontamination device according to any of Items DD1-DD58.

Item DS1. A decontamination system comprising:
a container having an inner volume defined by at least one wall including a first wall;
a carrier device rotatably arranged about a rotation axis in the inner volume;
a motor configured to rotate the carrier device;
a first port in the first wall, wherein the first port is configured to couple a decontamination device to the container; and
a second port in a wall of the container, wherein the second port is configured to couple a decontamination device to the container.

Item DS2. Decontamination system according to item DS1, wherein the first port comprises a bayonet coupling device for releasably coupling a decontamination device to the container.

Item DS3. Decontamination system according to any of items DS1-DS2, wherein the first port comprises a first closure element for closing a first opening of the first port.

Item DS4. Decontamination system according to any of items DS1-DS3, wherein the container is made of a UV absorbing polymer material.

Item DS5. Decontamination system according to any of items DS1-DS4, wherein the first port is configured to attach a decontamination device such that a first beam from the decontamination device coupled to the first port is in a first direction, and wherein the second port is configured to attach a decontamination device such that a second beam from the decontamination device coupled to the second port is in a second direction, wherein the first direction and the second direction form a beam angle larger than 30°.

Item DS6. Decontamination system according to item DS5, wherein a first angle between the rotation axis and the first direction is less than 60°.

Item DS7. Decontamination system according to any of items DS1-DS6, wherein a second angle between the rotation axis and the second direction is larger than 30°.

Item DS8. Decontamination system according to any of items DS1-DS7, wherein the container comprises a rotation opening, and wherein the carrier device comprises a shaft rotatably arranged in the rotation opening.

Item DS9. Decontamination system according to any of items DS1-DS8, wherein the carrier device comprises a frame part and a seat part, wherein the seat part comprises a seat configured for accommodating an item to be decontaminated, wherein the seat part is made of a seat material comprising silicone.

Item DS10. Decontamination system according to any of items DS1-DS9, the decontamination system comprising a first decontamination device and a second decontamination, wherein the first decontamination device is configured to releasably couple to the first port, and the second decontamination device is configured to releasably couple to the second port.

Item DS11. Decontamination system according to Item DS10, wherein the first decontamination device is a decontamination device according to any of Items DD1-DD58.

Item DS12. Decontamination system according to Item DS11, wherein the second decontamination device is a decontamination device according to any of Items DD1-DD58.

The use of the terms “first”, “second”, “third” and “fourth”, “primary”, “secondary”, “tertiary”, “quaternary”, “quinary”, “senary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary”, “quaternary”, “quinary”, “senary” etc. are used to distinguish one element from another. Note that the words “first”, “second”, “third” and “fourth”, “primary”, “secondary”, “tertiary”, “quaternary”, “quinary”, “senary” etc. are used here and elsewhere for labelling purposes only and are not intended to denote any specific spatial or temporal ordering. Furthermore, the labelling of a first element does not imply the presence of a second element and vice versa.

It may be appreciated that FIGS. 1-19 comprise some devices and/or elements or operations which are illustrated with a solid line and some devices and/or elements or operations which are illustrated with a dashed line. Devices and/or elements or operations which are comprised in a solid line are devices and/or elements or operations which are comprised in the broadest example embodiment. Devices and/or elements or operations which are comprised in a dashed line are example embodiments which may be comprised in, or a part of, or are further devices and/or elements or operations which may be taken in addition to devices and/or elements or operations of the solid line example embodiments. It should be appreciated that these operations need not be performed in order presented. Furthermore, it should be appreciated that not all of the operations need to be performed. The example operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

The various example methods, devices, and assemblies described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program circuitries may include routines, programs, objects, components, data structures, etc. that perform specified tasks or implement specific abstract data types. Computer-executable instructions, associated data structures, and program circuitries represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents.

### LIST OF REFERENCES

100, 100A, 100B, 100C decontamination device
102 housing
103A first end
103B second end
104 head part
106 handle part
106A first handle part
106B second handle part
106C third handle part
106D handle coating
108, 108A, 108B, 108C, 108D light source
110 light guide
111 UV light
112 UV-transparent area
114 interface, first interface
116 coupling device/coupling ring
116A rod/protrusion
116B recess
117 outer periphery of coupling device
118 heat sink
120 second interface
122 spacer plate
124 spacer ring
125 rotation ring element
126 electrical switch
128 power source/battery pack
130 light controller
132 context ring
134 connecting element
136 protection/end ring
138 input signal
140 control signal
140A first control signal
140B second control signal
140C third control signal
142 input detector
200A first primary light element, first primary UV-C LED
200B first secondary light element, first secondary UV-C LED
200C first tertiary light element, first tertiary UV-C LED
200D first quaternary light element, first quaternary UV-C LED
200E first quinary light element, first quinary UV-C LED
200F first senary light element, first senary UV-C LED
200G first septenary light element, first septenary UV-C LED,
201 circuit board
201A proximal surface of circuit board
202A second primary light element, second primary UV-A LED
202B second secondary light element, second secondary UV-A LED
202C second tertiary light element, second tertiary UV-A LED
202D second quaternary light element, second quaternary UV-A LED
202E second quinary light element, second quinary UV-A LED
202F second senary light element, second senary UV-A LED
204 third light element, third visible LED
204A third primary light element, third visible LED
204B third secondary light element, third visible LED
204C third tertiary light element, third visible LED
205 spacer
206 cut-out
208 alignment opening
300 reflector device
300A proximal surface of reflector device
300B distal surface of reflector device
302A first primary reflector cavity
302B first secondary reflector cavity
302C first tertiary reflector cavity
304C first quaternary reflector cavity
304A first primary reflector surface
304B first secondary reflector surface
304C first tertiary reflector surface
306A first primary reflector opening
306B first secondary reflector opening
306C first tertiary reflector opening
308A second primary reflector cavity
308B second secondary reflector cavity
308C second tertiary reflector cavity
310A second primary reflector surface
310B second secondary reflector surface
310C second tertiary reflector surface
312A second primary reflector opening
312B second secondary reflector opening
312C second tertiary reflector opening
314 third reflector cavity, center reflector cavity
316 third reflector surface
318 third reflector opening
320 alignment protrusion
322A first damping recess
322B second damping recess
322C third damping recess
350 alignment opening
370 alignment opening
372 first opening
400 decontamination system
402 container
404A first wall
404B second wall
404C third wall
406A port, first port
406B port, second port
406C port, third port
408A first direction
408B second direction
410 motor
412 rotation opening
414 shaft
416 carrier device
500 decontamination assembly
502, 503, 503A UV shield
502A first part
502B second part
504 coupling part
505 inner volume
505A wall of UV shield
506 rim
506A port
508 opening
510A first end
510B second end
512 reinforcement part
514 opening of port
600, 600A state diagram
602 first operating state
604 off state
606 lock state
608 inactive state
610 second operating state
620 on input detected
622 first input detected
624 stop input detected or first light scheme has been applied
626 stop input detected or first light scheme has been applied
628 off input detected
630 timer reaches a time threshold
632 second input detected
634 stop input detected or second light scheme has been applied
636 stop input detected or second light scheme has been applied
640 unlock input detected
642 off input detected
644 timer reaches a time threshold
T_1 duration of first light scheme
T_1_1 first primary time period
T_1_2 first secondary time period
T_2_1 second primary time period
T_2_2 second secondary time period
P_1_1 first primary pause
P_1_2 first secondary pause
O centre axis
X central axis
R rotation axis
α beam angle
α₁ first angle
α₂ second angle
H1 first height
H3 third height
R1 first radial distance

1. A decontamination device comprising:
a housing comprising a head part and a handle part;
a light source configured to emit UV-C light;
a light guide for guiding light from the light source towards a UV-transparent area in the head part of the housing; and
an interface,
wherein the light guide comprises a reflector device having a reflector structure in a proximal surface of the reflector device, the reflector structure comprising a plurality of reflector cavities comprising a first set of reflector cavities and a second set of reflector cavities, each reflector cavity having a reflector surface, the first set of reflector cavities including a first primary reflector cavity having a first primary reflector surface, and a first secondary reflector cavity having a first secondary reflector surface, and the second set of reflector cavities including a second primary reflector cavity having a second primary reflector surface.

2. Decontamination device according to claim 1, wherein the first primary reflector surface is a metallic surface.

3. Decontamination device according to claim 1, wherein the reflector device comprises a reflector body and a first layer coated onto the reflector body, wherein the reflector body is made of a body material and the first layer is made of a first reflector material, and wherein the first reflector material comprises a metal being one or more of aluminium, gold, copper, silver, and platinum.

4. Decontamination device according to claim 1, wherein the reflector surfaces are concave aspheric surfaces.

5. Decontamination device according to claim 1, wherein the reflector device comprises a first primary opening in the first primary reflector surface, wherein the first primary opening is circular with a diameter in the range from 2 mm to 15 mm.

6. Decontamination device according to claim 1, wherein the reflector structure comprises seven reflector cavities, wherein six reflector cavities including the first primary reflector cavity, the first secondary reflector cavity, and the second primary reflector cavity, are distributed around a third reflector cavity.

7. Decontamination device according to claim 1, wherein a center-to-center distance between two neighbouring reflector cavities is in the range from 10 mm to 25 mm.

8. Decontamination device according to claim 1, wherein the light source is configured to emit visible light and/or UV light comprising UV-A light.

9. Decontamination device according to claim 1, wherein the reflector device comprises a first recess and a second recess in the proximal surface of the reflector body, wherein the decontamination device comprises a first damping element at least partly accommodated in the first recess, and a second damping element at least partly accommodated in the second recess.

10. Decontamination device according to claim 1, wherein each reflector cavity has a depth in the range from 7 mm to 50 mm.

11. Decontamination device according to claim 1, wherein the light source comprises a circuit board and a first set of UV-C light emitting diodes mounted on the circuit board, the first set of UV-C light emitting diodes comprising at least three UV-C light emitting diodes.

12. Decontamination device according to claim 11, the decontamination device comprising a heat sink for cooling the light source, wherein the circuit board is made of material comprising a first layer of first material with a first thermal conductivity and a first heat capacity; and wherein the heat sink is in thermal contact with the first layer and made of a second material with a second thermal conductivity and a second heat capacity, wherein the second heat capacity is larger than the first heat capacity.

13. Decontamination device according to claim 1, wherein the decontamination device comprises a light controller connected to the light source for controlling the light source, wherein the interface is connected to the light controller, and wherein the light controller is configured to apply a first light scheme to the light source, wherein to apply the first light scheme comprises:
to activate a first set of UV-C light emitting diodes of the light source; and
to activate a second set of light emitting diodes of the light source, wherein the second set of light emitting diodes is configured to emit visible light and/or UV light comprising UV-A light.

14. Decontamination device according to claim 13, wherein the decontamination device is configured to operate in a plurality of states including a first operating state and one or more of a lock state, an inactive state, and a second operating state, the light controller comprising an input detector configured to detect a first input comprising a plurality of first input events and, wherein the light controller is configured to, in response to a detection of the first input, move the decontamination device to the first operating state.

15. A decontamination system comprising:
a decontamination device according to claim 1;
a container having an inner volume defined by at least one wall including a first wall;
a carrier device rotatably arranged about a rotation axis in the inner volume;
a motor configured to rotate the carrier device;
a first port in the first wall, wherein the first port is configured to couple the decontamination device to the container; and
a second port in a wall of the container, wherein the second port is configured to couple the decontamination device to the container.

16. A decontamination assembly comprising a decontamination device according to claim 1 and a UV shield, wherein the head part comprises a coupling device for releasably coupling the head part to a port of the UV shield.
